(19) United States
(12) Patent Application Publication
(10) Pub. No.: US 2024/0360088 A1
(43) Pub. Date: Oct. 31, 2024

(54) WATER-SOLUBLE NERVE DYES FOR IN VIVO NERVE-SPECIFIC IMAGING

(71) Applicant: Oregon Health & Science University, Portland, OR (US)

(72) Inventors: Summer L. Gibbs, Portland, OR (US); Lei G. Wang, Portland, OR (US); Connor W. Barth, Portland, OR (US); Antonio R. Montaño, Portland, OR (US)

(73) Assignee: Oregon Health & Science University, Portland, OR (US)

(21) Appl. No.: 18/575,219

(22) PCT Filed: Jul. 1, 2022
(86) PCT No.: PCT/US22/36015

§ 371 Date: Dec. 28, 2023

Related U.S. Application Data
(60) Provisional application No. 63/218,134, filed on Jul. 2, 2021.
(60) Provisional application No. 63/218,124, filed on Jul. 2, 2021.

(51) Int. Cl. C07D 265/38; A61K 49/00

(52) U.S. Cl. CPC C07D 265/38; A61K 49/0028; A61K 49/006

(57) ABSTRACT

Provided herein are libraries of nerve-specific fluorescent contrast agents of substituted 8-methyl-phenoxazine compounds of Formula (I) with excitation and emission profile comparable to US FDA approved methylene blue (MB) and indocyanine green (ICG), respectively, allowing for real-time intraoperative imaging using clinical-grade surgical systems. Also provided 700 nm lead candidates with substantially improved water-solubility, fully negating the need for formulation development with the added advantage of improved safety profiles for patient use in the clinic as well as decreased overall cost of clinical translation.

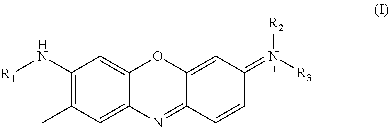

### CROSS-REFERENCE TO RELATED APPLICATIONS

This is the 371 National Phase of International Application No. PCT/US22/36015, filed Jul. 1, 2022, which claims priority to and the benefit of the earlier filing of U.S. Provisional Application No. 63/218,134, filed on Jul. 2, 2021; and of U.S. Provisional Application No. 63/218,124, filed on Jul. 2, 2021, each of which is incorporated by reference herein in its entirety.

### STATEMENT REGARDING FEDERALLY SPONSORED RESEARCH OR DEVELOPMENT

This invention was made with government support under grant R01EB021362 awarded by the National Institutes of Health. The government has certain rights in the invention.

### FIELD OF THE INVENTION

The present invention concerns nerve-specific fluorophore compounds useful in image-guided surgical techniques to avoid nerve tissues and decrease the morbidity of surgical procedures.

### BACKGROUND OF THE INVENTION

Fluorescence imaging in the NIR region (650-900 nm) is advantageous as endogenous tissue chromophore absorbance, scattering and autofluorescence are all at local minima, creating a black background upon which tissue-specific contrast can be added. The minimal photon scatter and absorbance in the NIR region also facilitates photon penetration for fluorescence imaging up to centimeters deep in tissue as compared to a few hundred microns using visible light.^{1,2} In the context of image-guided surgery, NIR fluorescence enables real-time, non-contact imaging where the addition of fluorescent contrast and NIR light does not alter the look of the surgical field and contrast agent detection does not require ionizing radiation.^{3,4} A limited number of fluorescent contrast agents exist that stain nerve tissue in vivo, none of which currently absorb or emit in the NIR region. There are currently seven known fluorophores that have been shown to have nerve or brain specificity, which include nerve-specific peptides and six classes of small molecule organic fluorophores. The nerve-specific peptides are a targeting sequence that largely bind to the epineurium with minimal binding to the endoneurium due to their large size, where binding to only the periphery of the nerve decreases its signal to background ratio (SBR) for in vivo nerve imaging.⁵ The six classes of small molecule organic fluorophores include a handful of stilbene derivatives,⁶ a coumarin analog,⁷ a library of distyrylbenzene (DSB) derivatives^{8,9} synthesized and characterized by our lab,^{10, 11} 8 styryl pyridinium (FM) fluorophores¹² characterized by our lab,¹³ an oxazine fluorophore, 14 and a tricarbocyanine (TCC) fluorophore.¹⁵ Although examples of nerve-specific small molecule organic fluorophores have been documented, their potential to provide NIR nerve-specific contrast is not equivalent (Table 1). The stilbene derivatives and coumarin analog inherently have ultraviolet (UV) excitation with blue emission, overlapping significantly with endogenous tissue chromophores, resulting in low nerve SBR.^{3,4} In our previous work, FM fluorophores had limited nerve specificity, highlighting only the dorsal nerve roots and the trigeminal ganglia when administered systemically.¹³ A library of DSB derivatives has been synthesized by our lab and utilized to determine the structure activity relationship of the DSB pharmacophore.¹⁰ To date our >200 synthesized DSB analogs demonstrate UV to blue excitation with green to red emission, where synthetic tuning to reach NIR emission is feasible, while NIR excitation is likely not possible while strictly maintaining the DSB core structure. A TCC fluorophore has been utilized for noninvasive brain imaging in hypo- and hypermyelinated mouse models demonstrating fluorescence correlation with myelination status.¹⁵ However preliminary fluorescence imaging studies in our lab have not shown nerve specificity at short time points (as suggested in the published study¹⁵) nor at longer time points used for previous nerve-specific fluorescence imaging studies by our lab.^{11,13} A red shifted oxazine fluorophore Oxazine 4 has demonstrated nerve-specific signal following systemic administration in all rodent nerves.¹⁴ However, it is not yet NIR fluorescent. There remains a need for improved agents for nerve-specific imaging.

### BRIEF SUMMARY OF THE INVENTION

We applied medicinal chemistry and modern organic synthesis to create several libraries of nerve-specific fluorescent contrast agents with excitation and emission profile comparable to US FDA approved methylene blue (MB) and indocyanine green (ICG), respectively, allowing for real-time intraoperative imaging using clinical-grade surgical systems. Additionally, we successfully engineered our 700 nm lead candidates with substantially improved water-solubility, fully negating the need for formulation development with the added advantage of improved safety profiles for patient use in the clinic as well as decreased overall cost of clinical translation.

An embodiment provides a compound of Formula (I):

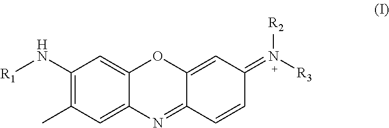

wherein:
R₁ is selected from the group of —X₁, —(CH₂)ₙ₁—SO₃, —(CH₂)ₙ₁—N⁺(CH₃)₃, —CH₂—CH₂—O—X₁, —CH₂—CH₂—O—[CH₂—CH₂—O]ₙ₄—X₁, —CH₂—CH₂—CH₂—O—X₁, and —CH₂—CH₂—CH₂—O—[CH₂—CH₂—CH₂—O]ₙ₅—X₁;

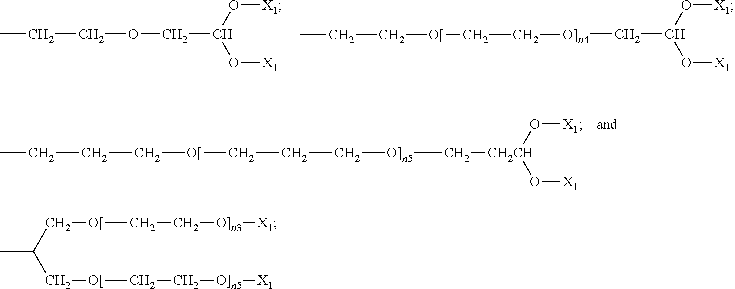

R₂ and R₅ are each independently selected from the group of methyl, ethyl, n-propyl, isopropyl, —(CH₂)ₙ₁—SO₃, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁;

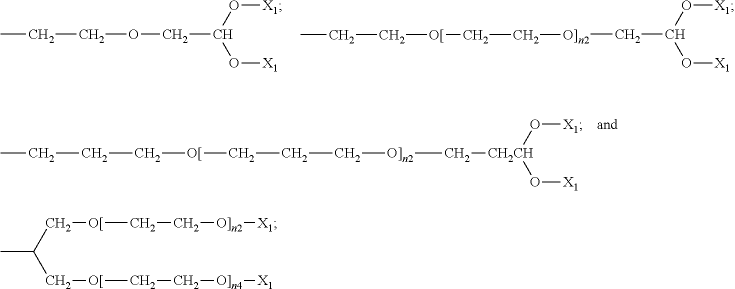

X₁ in each instance is independently selected from the group of C₁-C₆ straight or branched alkyl, C₂-C₆ straight or branched alkenyl, C₁-C₆ straight or branched alkynyl, and —Si(C₁-C₄ alkyl)₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
n5 is an integer independently selected in each instance from the group of 1, 2, 3, 4, 5, 6, 7, 8, 9, and 10;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

### DETAILED DESCRIPTION OF THE INVENTION

Another embodiment provides a compound of Formula (I), above, wherein R₁ is selected from the group of C₁-C₆ alkyl, —(CH₂)ₙ₁—SO₃, —(CH₂)ₙ₁—N⁺(CH₃)₃, —CH₂—CH₂—O—X₁, —CH₂—CH₂—O—[CH₂—CH₂—O]ₙ₄—X₁, —CH₂—CH₂—CH₂—O—X₁, and —CH₂—CH₂—CH₂—O—[CH₂—CH₂—CH₂—O]ₙ₅—X₁;

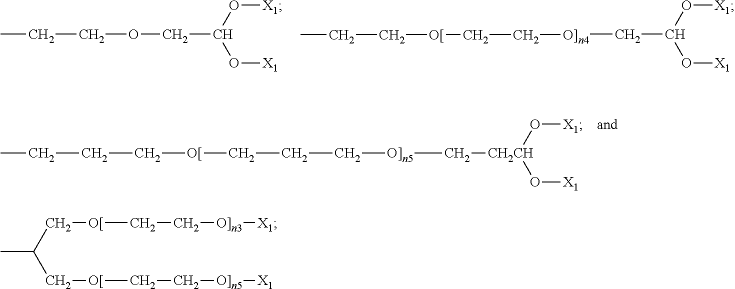

and the variables R₂, R₃, X₁, n1, n2, n3, n4, n5, are as defined, including provisos, as seen above for Formula (I).

Another embodiment provides a compound of Formula (I), above, wherein R₁ is selected from the group of C₁-C₄ alkyl, —(CH₂)ₙ₁—SO₃, —(CH₂)ₙ₁—N⁺(CH₃)₃, —CH₂—CH₂—O—X₁, —CH₂—CH₂—O—[CH₂—CH₂—O]ₙ₄—X₁, —CH₂—CH₂—CH₂—O—X₁, and —CH₂—CH₂—CH₂—O—[CH₂—CH₂—CH₂—O]ₙ₅—X₁;

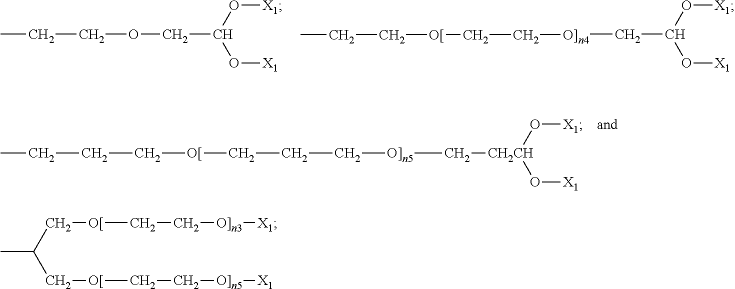

and the variables R₂, R₃, X₁, n1, n2, n3, n4, n5, are as defined, including provisos, as seen above for Formula (I).

Another embodiment provides a compound of Formula (I), above, wherein R₁ is selected from the group of —(CH₂)ₙ₁—SO₃, —(CH₂)ₙ₁—N⁺(CH₃)₃, —CH₂—CH₂—O—X₁, —CH₂—CH₂—O—[CH₂—CH₂—O]ₙ₄—X₁, —CH₂—CH₂—CH₂—O—X₁, and —CH₂—CH₂—CH₂—O—[CH₂—CH₂—CH₂—O]ₙ₅—X₁;

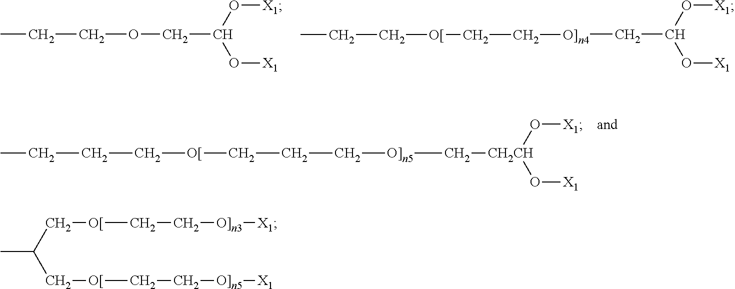

and the variables R₂, R₃, X₁, n1, n2, n3, n4, n5, are as defined, including provisos, as seen above for Formula (I).

Another embodiment provides a compound of Formula (I):

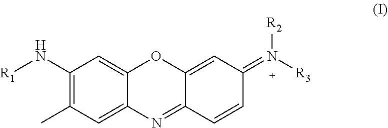

wherein:
R₁ is selected from the group of —X₁, —(CH₂)ₙ₁—SO₃, —(CH₂)ₙ₁—N⁺(CH₃)₃;
—CH₂—CH₂—O—X₁;
—CH₂—CH₂—O—[CH₂—CH₂—O]ₙ₄—X₁;
—CH₂—CH₂—CH₂—O—X₁; and
—CH₂—CH₂—CH₂—O—[CH₂—CH₂—CH₂—O]ₙ₅—X₁; R₂ and R₅ are each independently selected from the group of methyl, ethyl, n-propyl, isopropyl, —(CH₂)ₙₜ—SO₃,
—(CH₂)ₙ₁—N⁺(CH₃)₃;
—CH₂—CH₂—O—X₁;
—CH₂—CH₂—O—[CH₂—CH₂—O]ₙ₂—X₁;
—CH₂—CH₂—CH₂—O—X₁; and
—CH₂—CH₂—CH₂—O—[CH₂—CH₂—CH₂—O]ₙ₃—X₁;
X₁ in each instance is independently selected from the group of C₁-C₆ straight or branched alkyl, C₂-C₆ straight or branched alkenyl, C₁-C₆ straight or branched alkynyl, and —Si(C₁-C₄ alkyl)₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
n5 is an integer independently selected in each instance from the group of 1, 2, 3, 4, 5, 6, 7, 8, 9, and 10;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

A further embodiment provides a compound of Formula (I), wherein:
R₁ is selected from the group of —X₁, —(CH₂)ₙ₁—SO₃, —(CH₂)ₙ₁—N⁺(CH₃)₃;
—CH₂—CH₂—O—CH₃;
—CH₂—CH₂—O—CH₂—CH₃, —CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
R₂ and R₅ are each independently selected from the group of methyl, ethyl, n-propyl, isopropyl, —(CH₂)ₙₜ—SO₃, —(CH₂)ₙ₁—N⁺(CH₃)₃;
—CH₂—CH₂—O—CH₃;
—CH₂—CH₂—O—CH₂—CH₃, —CH₂—CH₂—O—[CH₂—CH₂—O]ₙ₂—CH₃;
—CH₂—CH₂—O—[CH₂—CH₂—O]ₙ₂—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₃—CH₃; and
—CH₂—CH₂—CH₂—O—[CH₂—CH₂—CH₂—O]ₙ₃—CH₂—CH₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
n5 is an integer independently selected in each instance from the group of 1, 2, 3, 4, 5, 6, 7, 8, 9, and 10;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

A further embodiment provides a compound of Formula (I), wherein:
R₁ is selected from the group of —X₁, —(CH₂)ₙ₁—SO₃, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
R₂ and R₃ are each independently selected from the group of methyl, ethyl, n-propyl, isopropyl, —(CH₂)ₙ₁—SO₃, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₂—CH₃;
—CH₂—CH₂—O—[CH₂—CH₂—O]ₙ₂—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₃—CH₃; and
—CH₂—CH₂—CH₂—O—[CH₂—CH₂—CH₂—O]ₙ₃—CH₂—CH₃;
n1 is an integer independently selected in each instance from the group of 1, 2, 3, and 4;
n2 is an integer independently selected in each instance from the group of 1, 2, 3, 4, 5, 6, 7, and 8;
n3 is an integer independently selected in each instance from the group of 1, 2, 3, 4, 5, 6, 7, and 8;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

A further embodiment provides a compound of Formula (I), wherein:
R₁ is selected from the group of —X₁, —(CH₂)ₙ₁—SO₃, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
R₂ is selected from the group of methyl, ethyl, n-propyl, isopropyl, —(CH₂)ₙ₁—SO₃, —(CH₂)ₙ₁—N⁺(CH₃)₃;
R₃ is selected from the group of methyl, ethyl, n-propyl, isopropyl, —(CH₂)ₙ₁—SO₃, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₂—CH₃;
—CH₂—CH₂—O—[CH₂—CH₂—O]ₙ₂—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₃—CH₃; and
—CH₂—CH₂—CH₂—O—[CH₂—CH₂—CH₂—O]ₙ₃—CH₂—CH₃;
n1 is an integer independently selected in each instance from the group of 1, 2, 3, and 4;
n2 is an integer independently selected in each instance from the group of 1, 2, 3, 4, 5, 6, 7, and 8;
n3 is an integer independently selected in each instance from the group of 1, 2, 3, 4, 5, 6, 7, and 8;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

Yet another embodiment provides a compound of Formula (I), wherein:
R₁, R₂, and R₃ are each selected from the group of —(CH₂)ₙ₁—SO₃, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
n1 is an integer independently selected in each instance from the group of 1, 2, 3, and 4;
n2 is an integer independently selected in each instance from the group of 1, 2, 3, 4, 5, 6, 7, and 8;
n3 is an integer independently selected in each instance from the group of 1, 2, 3, 4, 5, 6, 7, and 8;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

A different embodiment provides a compound of Formula (I), wherein:
R₁, R₂, and R₃ are each selected from the group of:
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
n1 is an integer independently selected in each instance from the group of 1, 2, 3, and 4;
n2 is an integer independently selected in each instance from the group of 1, 2, 3, 4, 5, 6, 7, and 8;
n3 is an integer independently selected in each instance from the group of 1, 2, 3, 4, 5, 6, 7, and 8;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

An additional embodiment provides a compound of Formula (I), wherein:
R₁ is selected from the group of:
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
R₂ is selected from the group of methyl, ethyl, n-propyl, and isopropyl; R₃ is selected from the group of methyl, ethyl, n-propyl, and isopropyl;
—CH₂—CH₂—O—CH₃;
—CH₂—CH₂—O—CH₂—CH₃,
—CH₂—CH₂—O—[CH₂—CH₂—O]ₙ₂—CH₃;
—CH₂—CH₂—O—[CH₂—CH₂—O]ₙ₂—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₃—CH₃; and
—CH₂—CH₂—CH₂—O—[CH₂—CH₂—CH₂—O]ₙ₃—CH₂—CH₃;
n1 is an integer independently selected in each instance from the group of 1, 2, 3, and 4;
n2 is an integer independently selected in each instance from the group of 1, 2, 3, 4, 5, 6, 7, and 8;
n3 is an integer independently selected in each instance from the group of 1, 2, 3, 4, 5, 6, 7, and 8;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

An additional embodiment provides a compound of Formula (I), wherein:
R₁ is selected from the group of:
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
R₂ and R₅ are each independently selected from the group of methyl, ethyl, n-propyl, and isopropyl;
n1 is an integer independently selected in each instance from the group of 1, 2, 3, and 4;
n2 is an integer independently selected in each instance from the group of 1, 2, 3, 4, 5, 6, 7, and 8;
n3 is an integer independently selected in each instance from the group of 1, 2, 3, 4, 5, 6, 7, and 8;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

Still another embodiment provides a compound of Formula (II):

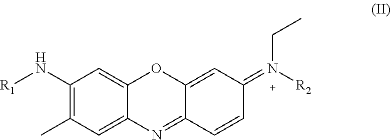

wherein:
R₁ and R₂ are each independently selected from the group of —(CH₂)ₙ₁—SO₃, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
n1 is an integer independently selected in each instance from the group of 1, 2, 3, and 4;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8.

An additional embodiment provides a compound of Formula (III):

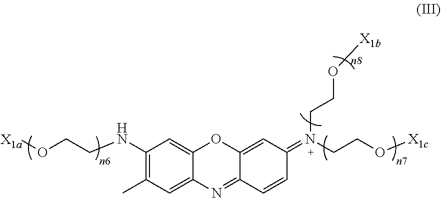

wherein:
X₁ₐ, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6, n7, and n8 are each an integer independently selected in each instance from the group of 1, 2, 3, 4, and 5.

Another embodiment provides a compound of Formula (III), above, wherein:
X₁ₐ, X_{1b}, and X_{1c} are each independently selected from the group of C₁-C₆ straight or branched alkyl; and
   n6, n7, and n8 are each an integer independently selected in each instance from the group of 1, 2, 3, 4, and 5.

Another embodiment provides a compound of Formula (III), above, wherein:
X₁ₐ, X_{1b}, and X_{1c} are each independently selected from the group of C₁-C₄ straight or branched alkyl; and
   n6, n7, and n8 are each an integer independently selected in each instance from the group of 1, 2, 3, 4, and 5.

Another embodiment provides a compound of Formula (III), above, wherein:
X₁ₐ, X_{1b}, and X_{1c} are each independently selected from the group of C₁-C₆ straight or branched alkyl; and
   n6, n7, and n8 are each an integer independently selected in each instance from the group of 1, 2, 3, and 4.

Another embodiment provides a compound of Formula (III), above, wherein:
X₁ₐ, X_{1b}, and X_{1c} are each independently selected from the group of C₁-C₄ straight or branched alkyl; and
   n6, n7, and n8 are each an integer independently selected in each instance from the group of 1, 2, 3, and 4.

Still another embodiment provides a compound of Formula (III), above, wherein:
X₁ₐ, X_{1b}, and X_{1c} are each independently selected from the group of C₁-C₆ straight or branched alkyl; and
   n6, n7, and n8 are each an integer independently selected in each instance from the group of 1, 2, and 3.

Still another embodiment provides a compound of Formula (III), above, wherein:
X₁ₐ, X_{1b}, and X_{1c} are each independently selected from the group of C₁-C₄ straight or branched alkyl; and
   n6, n7, and n8 are each an integer independently selected in each instance from the group of 1, 2, and 3.

Another embodiment provides a compound of Formula (IV):

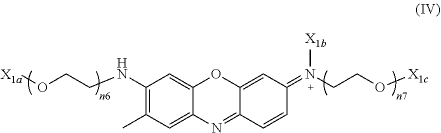

wherein:
X₁a, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6 and n7 are each an integer independently selected in each instance from the group of 1, 2, 3, 4, and 5.

Another embodiment provides a compound of Formula (IV), above, wherein:
X₁a, X_{1b}, and X_{1c} are each independently selected from the group of C₁-C₆ straight or branched alkyl; and
n6 and n7 are each an integer independently selected in each instance from the group of 1, 2, 3, 4, and 5.

Another embodiment provides a compound of Formula (IV), above, wherein:
X₁a, X_{1b}, and X_{1c} are each independently selected from the group of C₁-C₄ straight or branched alkyl; and
n6 and n7 are each an integer independently selected in each instance from the group of 1, 2, 3, 4, and 5.

A different embodiment provides a compound of Formula (IV), above, wherein:
X₁ₐ, X_{1b}, and X_{1c} are each independently selected from the group of C₁-C₆ straight or branched alkyl; and
n6 and n7 are each an integer independently selected in each instance from the group of 1, 2, 3, and 4.

Another different embodiment provides a compound of Formula (IV), above, wherein:
X₁a, X_{1b}, and X_{1c} are each independently selected from the group of C₁-C₄ straight or branched alkyl; and
n6 and n7 are each an integer independently selected in each instance from the group of 1, 2, 3, and 4.

A further embodiment provides a compound of Formula (IV), above, wherein:
X₁a, X_{1b}, and X_{1c} are each independently selected from the group of C₁-C₆ straight or branched alkyl; and
n6 and n7 are each an integer independently selected in each instance from the group of 1, 2, and 3.

A still further embodiment provides a compound of Formula (IV), above, wherein:
X₁a, X_{1b}, and X_{1c} are each independently selected from the group of C₁-C₄ straight or branched alkyl; and
n6 and n7 are each an integer independently selected in each instance from the group of 1, 2, and 3.

An additional embodiment provides a compound of Formula (V):

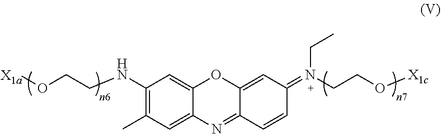

wherein:
X₁a and X_{1b} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6 and n7 are each an integer independently selected in each instance from the group of 1, 2, 3, 4, and 5.

Another embodiment provides a compound of Formula (V), above, wherein:
X₁ₐ and X_{1b} are each independently selected from the group of C₁-C₆ straight or branched alkyl; and
n6 and n7 are each an integer independently selected in each instance from the group of 1, 2, 3, 4, and 5.

Another embodiment provides a compound of Formula (V), above, wherein:
X₁a and X_{1b} are each independently selected from the group of C₁-C₄ straight or branched alkyl; and
n6 and n7 are each an integer independently selected in each instance from the group of 1, 2, 3, 4, and 5.

Another embodiment provides a compound of Formula (V), above, wherein:
X₁a and X_{1b} are each independently selected from the group of C₁-C₆ straight or branched alkyl; and
n6 and n7 are each an integer independently selected in each instance from the group of 1, 2, 3, and 4.

Another embodiment provides a compound of Formula (V), above, wherein:
X₁a and X_{1b} are each independently selected from the group of C₁-C₄ straight or branched alkyl; and
n6 and n7 are each an integer independently selected in each instance from the group of 1, 2, 3, and 4.

Another embodiment provides a compound of Formula (V), above, wherein:
X₁a and X_{1b} are each independently selected from the group of C₁-C₆ straight or branched alkyl; and
n6 and n7 are each an integer independently selected in each instance from the group of 1, 2, and 3.

Another embodiment provides a compound of Formula (V), above, wherein:
X₁a and X_{1b} are each independently selected from the group of C₁-C₄ straight or branched alkyl; and
n6 and n7 are each an integer independently selected in each instance from the group of 1, 2, and 3.

An additional embodiment provides a compound of Formula (VI):

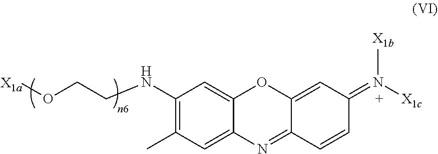

X₁ₐ, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6 is an integer selected from the group of 1, 2, 3, 4, and 5.

Another embodiment provides a compound of Formula (VI), above, wherein:
X₁ₐ, X_{1b}, and X_{1c} are each independently selected from the group of C₁-C₆ straight or branched alkyl; and
n6 is an integer selected from the group of 1, 2, 3, 4, and 5.

Another embodiment provides a compound of Formula (VI), above, wherein:
X₁ₐ, X_{1b}, and X_{1c} are each independently selected from the group of C₁-C₄ straight or branched alkyl; and
n6 is an integer selected from the group of 1, 2, 3, 4, and 5.

Another embodiment provides a compound of Formula (VI), above, wherein:
X₁ₐ, X_{1b}, and X_{1c} are each independently selected from the group of C₁-C₆ straight or branched alkyl; and
n6 is an integer selected from the group of 1, 2, 3, and 4.

Another embodiment provides a compound of Formula (VI), above, wherein:
X₁ₐ, X_{1b}, and X_{1c} are each independently selected from the group of C₁-C₄ straight or branched alkyl; and
n6 is an integer selected from the group of 1, 2, 3, and 4.

Another embodiment provides a compound of Formula (VI), above, wherein:
X₁ₐ, X_{1b}, and X_{1c} are each independently selected from the group of C₁-C₆ straight or branched alkyl; and
n6 is an integer selected from the group of 1, 2, and 3.

Another embodiment provides a compound of Formula (VI), above, wherein:
X₁ₐ, X_{1b}, and X_{1c} are each independently selected from the group of C₁-C₄ straight or branched alkyl; and
n6 is an integer selected from the group of 1, 2, and 3.

An additional embodiment provides a compound of Formula (VII):

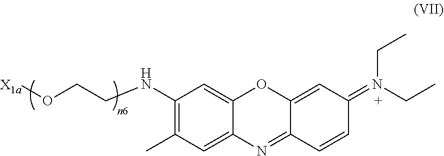

X₁ₐ is selected from the group of C₁-C₆ straight or branched alkyl, C₂-C₆ straight or branched alkenyl, C₁-C₆ straight or branched alkynyl, and —Si(C₁-C₄ alkyl)₃; and
n6 is an integer selected from the group of 1, 2, 3, 4, and 5.

Another embodiment provides a compound of Formula (VII), above, wherein:
X₁ₐ is C₁-C₆ straight or branched alkyl; and
n6 is an integer selected from the group of 1, 2, 3, 4, and 5.

Another embodiment provides a compound of Formula (VII), above, wherein:
X₁ₐ is C₁-C₄ straight or branched alkyl; and
n6 is an integer selected from the group of 1, 2, 3, 4, and 5.

Another embodiment provides a compound of Formula (VII), above, wherein:
X₁ₐ is C₁-C₆ straight or branched alkyl; and
n6 is an integer selected from the group of 1, 2, 3, and 4.

Another embodiment provides a compound of Formula (VII), above, wherein:
X₁ₐ is C₁-C₄ straight or branched alkyl; and
n6 is an integer selected from the group of 1, 2, 3, and 4.

Another embodiment provides a compound of Formula (VII), above, wherein:
X₁a is C₁-C₆ straight or branched alkyl; and
n6 is an integer selected from the group of 1, 2, and 3.

Another embodiment provides a compound of Formula (VII), above, wherein:
X₁ₐ is C₁-C₄ straight or branched alkyl; and
n6 is an integer selected from the group of 1, 2, and 3.

An additional embodiment provides a compound of Formula (VIII):

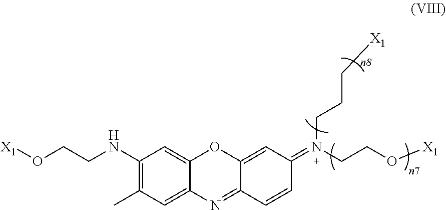

wherein:
X₁ₐ, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n7 and n8 are each an integer independently selected in each instance from the group of 1, 2, 3, 4, and 5.

An additional embodiment provides a compound of Formula (VIII), above, wherein:
X₁a, X_{1b}, and X_{1c} are each independently selected from the group of C₁-C₆ straight or branched alkyl; and
n7 and n8 are each an integer independently selected in each instance from the group of 1, 2, 3, 4, and 5.

An additional embodiment provides a compound of Formula (VIII), above, wherein:
X₁a, X_{1b}, and X_{1c} are each independently selected from the group of C₁-C₄ straight or branched alkyl; and
n7 and n8 are each an integer independently selected in each instance from the group of 1, 2, 3, 4, and 5.

Another embodiment provides a compound of Formula (VIII), above, wherein:
X₁ₐ, X_{1b}, and X_{1c} are each independently selected from the group of C₁-C₆ straight or branched alkyl; and
n7 and n8 are each an integer independently selected in each instance from the group of 1, 2, 3, and 4.

Another embodiment provides a compound of Formula (VIII), above, wherein:
X₁a, X_{1b}, and X_{1c} are each independently selected from the group of C₁-C₄ straight or branched alkyl; and
n7 and n8 are each an integer independently selected in each instance from the group of 1, 2, 3, and 4.

A further embodiment provides a compound of Formula (VIII), above, wherein:
X₁a, X_{1b}, and X_{1c} are each independently selected from the group of C₁-C₆ straight or branched alkyl; and
n7 and n8 are each an integer independently selected in each instance from the group of 1, 2, and 3.

A further embodiment provides a compound of Formula (VIII), above, wherein:
X₁a, X_{1b}, and X_{1c} are each independently selected from the group of C₁-C₄ straight or branched alkyl; and
n7 and n8 are each an integer independently selected in each instance from the group of 1, 2, and 3.

Another embodiment provides a compound of Formula (IX):

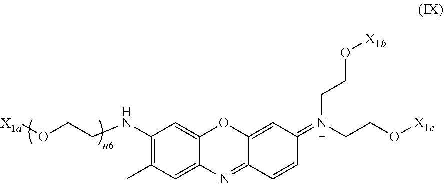

wherein:
X₁a, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6 is an integer independently selected from the group of 1, 2, 3, 4, and 5.

A further embodiment provides a compound of Formula (IX), above, wherein:
X₁a, X_{1b}, and X_{1c} are each independently selected from the group of C₁-C₆ straight or branched alkyl; and
n6 is an integer independently selected from the group of 1, 2, 3, 4, and 5.

A further embodiment provides a compound of Formula (IX), above, wherein:
X₁a, X_{1b}, and X_{1c} are each independently selected from the group of C₁-C₄ straight or branched alkyl; and
n6 is an integer independently selected from the group of 1, 2, 3, 4, and 5.

A further embodiment provides a compound of Formula (IX), above, wherein:
X₁a, X_{1b}, and X_{1c} are each independently selected from the group of C₁-C₆ straight or branched alkyl; and
n6 is an integer independently selected from the group of 1, 2, 3, and 4.

A further embodiment provides a compound of Formula (IX), above, wherein:
X₁ₐ, X_{1b}, and X_{1c} are each independently selected from the group of C₁-C₄ straight or branched alkyl; and
n6 is an integer independently selected from the group of 1, 2, 3, and 4.

A still further embodiment provides a compound of Formula (IX), above, wherein:
X₁a, X_{1b}, and X_{1c} are each independently selected from the group of C₁-C₆ straight or branched alkyl; and
n6 is an integer independently selected from the group of 1, 2, and 3.

A still further embodiment provides a compound of Formula (IX), above, wherein:
X₁ₐ, X_{1b}, and X_{1c} are each independently selected from the group of C₁-C₄ straight or branched alkyl; and
n6 is an integer independently selected from the group of 1, 2, and 3.

A further embodiment provides a compound of Formula (X):

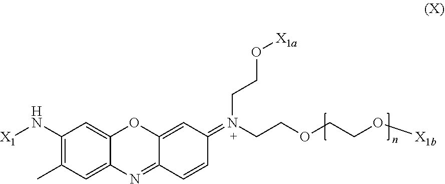

wherein:
n is an integer selected from 2, 3, 4, and 5;
X₁ is C₁-C₆ alkyl; and
X₁ₐ and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

A further embodiment provides a compound of Formula (X), above, wherein n is an integer selected from 2, 3, 4, and 5;
X₁ is C₁-C₄ alkyl; and
X₁ₐ and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

A further embodiment provides a compound of Formula (X), above, wherein n is an integer selected from 2, 3, 4, and 5;
X₁ is C₁-C₂ alkyl; and
X₁a and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

A further embodiment provides a compound of Formula (X), above, wherein n is an integer selected from 2, 3, 4, and 5;
X₁ is C₂-C₄ alkyl; and
X₁ₐ and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

A further embodiment provides a compound of Formula (X), above, wherein n is an integer selected from 2, 3, 4, and 5;
X₁ is C₂-C₃ alkyl; and
X₁ₐ and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

A further embodiment provides a compound of Formula (XI):

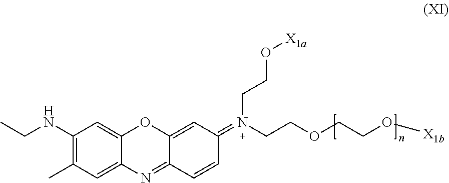

wherein:
n is an integer selected from 2, 3, 4, and 5; and
X₁a and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

Another embodiment provides a compound of Formula (XI), wherein:
n is an integer selected from 2, 3, and 4; and
X₁a and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

A further embodiment provides a compound of Formula (XI), wherein:
n is an integer selected from 2 and 3; and
X₁ₐ and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

A still further embodiment provides a compound of Formula (XI), wherein:
n is 2; and
X₁ₐ and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

A still further embodiment provides a compound of Formula (XI), wherein:
n is 3; and
X₁a and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

### Group 2

Another embodiment provides a compound of Formula (XI), wherein:
n is an integer selected from 2, 3, and 4; and
X₁a and X_{1b} are each selected independently from the group of C₁-C₄ straight or branched alkyl, C₂-C₄ straight or branched alkenyl, C₁-C₄ straight or branched alkynyl, and —Si(C₁-C₄ alkyl)₃.

A further embodiment provides a compound of Formula (XI), wherein:
n is an integer selected from 2 and 3; and
X₁a and X_{1b} are each selected independently from the group of C₁-C₄ straight or branched alkyl, C₂-C₄ straight or branched alkenyl, C₁-C₄ straight or branched alkynyl, and —Si(C₁-C₄ alkyl)₃.

A still further embodiment provides a compound of Formula (XI), wherein:
n is 2; and
X₁a and X_{1b} are each selected independently from the group of C₁-C₄ straight or branched alkyl, C₂-C₄ straight or branched alkenyl, C₁-C₄ straight or branched alkynyl, and —Si(C₁-C₄ alkyl)₃.

A still further embodiment provides a compound of Formula (XI), wherein:
n is 3; and
X₁ₐ and X_{1b} are each selected independently from the group of C₁-C₄ straight or branched alkyl, C₂-C₄ straight or branched alkenyl, C₁-C₄ straight or branched alkynyl, and —Si(C₁-C₄ alkyl)₃.

### Group 3

Another embodiment provides a compound of Formula (XI), wherein:
n is an integer selected from 2, 3, and 4; and
X₁ₐ and X_{1b} are each selected independently from the group of C₁-C₃ straight or branched alkyl, C₂-C₃ alkenyl, C₁-C₃ alkynyl, and —Si(C₁-C₃ alkyl)₃.

A further embodiment provides a compound of Formula (XI), wherein:
n is an integer selected from 2 and 3; and
X₁a and X_{1b} are each selected independently from the group of C₁-C₃ straight or branched alkyl, C₂-C₃ alkenyl, C₁-C₃ alkynyl, and —Si(C₁-C₃ alkyl)₃.

A still further embodiment provides a compound of Formula (XI), wherein:
n is 2; and
X₁ₐ and X_{1b} are each selected independently from the group of C₁-C₃ straight or branched alkyl, C₂-C₃, C₁-C₃ alkynyl, and —Si(C₁-C₃ alkyl)₃.

A still further embodiment provides a compound of Formula (XI), wherein:
n is 3; and
X₁a and X₁₆ are each selected independently from the group of C₁-C₃ straight or branched alkyl, C₂-C₃, C₁-C₃ alkynyl, and —Si(C₁-C₃ alkyl)₃.

### Group 4

Another embodiment provides a compound of Formula (XI), wherein:
n is an integer selected from 2, 3, and 4; and
X₁a and X_{1b} are each selected independently from the group of C₁-C₂ alkyl, ethenyl, ethynyl, and —Si(C₁-C₃ alkyl)₃.

A further embodiment provides a compound of Formula (XI), wherein:
n is an integer selected from 2 and 3; and
X₁a and X_{1b} are each selected independently from the group of C₁-C₂ alkyl, ethenyl, ethynyl, and —Si(C₁-C₃ alkyl)₃.

A still further embodiment provides a compound of Formula (XI), wherein:
n is 2; and
X₁ₐ and X_{1b} are each selected independently from the group of C₁-C₂ alkyl, ethenyl, ethynyl, and —Si(C₁-C₃ alkyl)₃.

A still further embodiment provides a compound of Formula (XI), wherein:
n is 3; and
X₁ₐ and X_{1b} are each selected independently from the group of C₁-C₂ alkyl, ethenyl, ethynyl, and —Si(C₁-C₃ alkyl)₃.

Yet another embodiment provides a compound of Formula (XII):

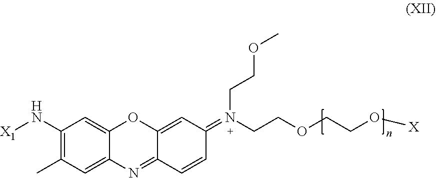

n is an integer selected from 2, 3, and 4;
X₁ is C₁-C₆ alkyl; and
X is selected from the group of C₁-C₆ straight or branched alkyl, C₂-C₆ straight or branched alkenyl, C₁-C₆ straight or branched alkynyl, and —Si(C₁-C₄ alkyl)₃.

Yet another embodiment provides a compound of Formula (XIII):

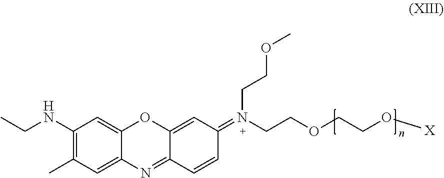

n is an integer selected from 2, 3, and 4; and
X is selected from the group of C₁-C₆ straight or branched alkyl, C₂-C₆ straight or branched alkenyl, C₁-C₆ straight or branched alkynyl, and —Si(C₁-C₄ alkyl)₃.

Another embodiment provides a compound of Formula (XIII), wherein:
n is an integer selected from 2 and 3; and
is selected from the group of C₁-C₆ straight or branched alkyl, C₂-C₆ straight or branched alkenyl, C₁-C₆ straight or branched alkynyl, and —Si(C₁-C₄ alkyl)₃.

Another embodiment provides a compound of Formula (XIII), wherein:
n is an integer selected from 2 and 3; and
is selected from the group of C₁-C₄ straight or branched alkyl, C₂-C₄ straight or branched alkenyl, C₁-C₄ straight or branched alkynyl, and —Si(C₁-C₄ alkyl)₃.

Another embodiment provides a compound of Formula (XIII), wherein:
n is an integer selected from 2 and 3; and
is selected from the group of C₁-C₃ straight or branched alkyl, C₂-C₃₄ alkenyl, C₁-C₃ alkynyl, and —Si(C₁-C₄ alkyl)₃.

Another embodiment provides a compound of Formula (XIII), wherein:
n is an integer selected from 2 and 3; and
X is C₁-C₃ straight or branched alkyl.

Another embodiment provides a compound of Formula (XIII), wherein:
n is 2; and
X is C₁-C₃ straight or branched alkyl.

Another embodiment provides a compound of Formula (XIII), wherein:
n is 3; and
X is C₁-C₃ straight or branched alkyl.

### Definitions

A “subject” or a “patient” refers to any animal. The animal may be a mammal. Examples of suitable mammals include human and non-human primates, dogs, cats, sheep, cows, pigs, horses, mice, rats, rabbits, and guinea pigs. In some embodiments the subject or patient is a human, particularly including a human undergoing or in need of a surgical procedure or examination.

The term “nerve” used herein means a bundle of neural axons. Within a nerve, each axon is surrounded by a layer of connective tissue called the endoneurium. The axons are bundled together into groups called fascicles, and each fascicle is wrapped in a layer of connective tissue called the perineurium. The entire nerve is wrapped in a layer of connective tissue called the epineurium. The term “nerve” is intended to include any tissues (e.g., the sinoatrial node or the atrioventricular node) or structures associated therewith (e.g., neuromuscular junctions).

The term “nerve-specific” or “nerve specific” herein refers to an agent that is drawn to a nerve or nerve tissue and may be used in fluorescent imaging techniques to help contrast and differentiate the nerve or nerve tissue from surrounding cells and/or tissues. The term “nerve specificity” refers to the nature or activity of an agent being nerve-specific.

The term “near infrared” or the acronym “(NIR)” refers to light at the near infrared spectrum, generally at a wavelength of about 0.65 to about 1.4 μm (700 nm-1400 nm. It may also refer to a range designated by the International Organization for Standardization as from a wavelength of about 0.78 μm to about 3 μm. In some embodiments, the preferred near infrared spectroscopy and imaging (NIRS) range is from about 650 nm to about 950 nm. In other embodiments, the preferred near infrared spectroscopy and imaging (NIRS) range is from about 650 nm to about 900 nm.

In some embodiments the agents and/or compositions comprising them are intended for direct/topical administration. Direct or topical administration are understood herein to comprise the administration of an agent or composition directly to surface of a tissue, organ, nerve bundle, or other bodily component. In some methods, the administration may be accomplished by brushing, spraying, or irrigation with the appropriate compound or composition.

In other embodiments, the agents and/or compositions may be administered systemically to the patient or subject, such as through intravenous injection or infusion.

In other embodiments, the agents and/or compositions may be administered locally to a desired tissue or organ, such as through injection.

The terms “effective amount” or “medically effective amount” or like terms refers to an amount of a compound or composition as described herein to cover a target area sufficiently to complete binding to one or more nerves such that they may be identified through relevant imaging techniques, particularly near-infrared imaging techniques.

The term “imaging” herein refers to the use of fluorescent compounds in conventional medical imaging techniques including, but not limited to, those related to fluorescence image-guided surgery (including minimally invasive laparoscopy or endoscopy techniques), computer-assisted surgery or surgical navigation, radiosurgery or radiation therapy, interventional radiology, fluorescence microscopy, and laser-confocal microscopy. These techniques may include near infrared wavelengths from about 650 nm to 900 nm.

The term “label” refers to a molecule that facilitates the visualization and/or detection of a targeting molecule disclosed herein. In some embodiments, the label is a fluorescent moiety. The term “labeling” refers to a successful administration of the label to a target to allow such detection.

As used herein, the terms “robotic surgery”, “robot-assisted surgery”, or “computer-assisted surgery” refer to surgical techniques involving robotic systems that control the movement of medical instruments to conduct a surgical procedure with precise, flexible, and/or minimally invasive actions designed to limit the amount of surgical trauma, blood loss, pain, scarring, and post-surgical patient recovery time and/or complications, such as infection at the surgical area. Examples of robotic surgery include those conducted using the da Vinci Surgical System (Intuitive Surgical, Sunnyvale, CA, USA) approved by the U.S. Food and Drug Administration in 2000.

The terms “surgery” or “surgical method” as used herein, refers to any method used to manipulate, change, or cause an effect by a physical intervention. These methods include, but are not limited to open surgery, endoscopic surgery, laparoscopic surgery, minimally invasive surgery, robotic surgery, any procedures that may affect any neuron or nerve, such as placement of retractors during spinal surgery, electrically conducting cardiac tissue or nerve ablation, epidural injection, intrathecal injections, neuron or nerve blocks, implantation of devices such as neuron or nerve stimulators and implantation of pumps. These methods may also include biopsy or other invasive techniques for the collection of cell or tissue samples, such as for diagnostic purposes.

As used herein, the term “targeting molecule” refers to any agent (e.g., peptide, protein, nucleic acid polymer, aptamer, or small molecule) that associates with (e.g., binds to) a target of interest. The target of interest may be a nerve cell or an organ or tissue associated with one or more nerve cells or nerve structures. In some embodiments, the targeting molecule is any agent that associates with (e.g., binds to) a target comprising one or more neurons, nerves, or tissues or structures associated therewith, i.e. nerve tissues, nervous system tissues, nerve bundles, etc. It is understood that nerve and nerve-related targets include those associated with the brain and spinal cord of the central nervous system (CNS) and the nerves of the peripheral nervous system (PNS).

The term “prostatectomy” refers to a surgical technique to remove all or part of a subject's prostate gland. A “radical prostatectomy” concerns removal of a subject's entire prostate gland, along with surrounding tissues, often including the seminal vesicles and nearby lymph nodes.

The terms “orthopedic limb repair” or “orthopedic limb repair surgeries” refer to surgical techniques performed on the limb musculoskeletal system of a subject. These techniques include limb reconstruction surgeries, joint replacement procedures, revision joint surgery, debridement, bone fusions, tendon or ligament repair, internal fixation of bone, and osteotomies.

The term “fluorophore” herein refers to any one of the compounds described herein for use in imaging techniques, particularly for nerve imaging techniques. Each of the compounds described herein as the product of a specific synthesis or described in a generic description is considered fluorophore for methods, uses, and compositions.

The term “variable” or “variables” used in the generic descriptions and claims herein refer to the entities or moieties that may, in some instances, be chosen from a specified group. Such variables may include R, R₁, R₂, n1, n2, n3, n4, n5, X₁, and the like.

All ranges disclosed and/or claimed herein are inclusive of the recited endpoint and independently combinable (for example, the ranges of “from 2 to 10” and “2-10” are inclusive of the endpoints, 2 and 10, and all the intermediate values).

The term “intraoperatively” as used in describing methods or uses herein refers to an activity that occurs during a surgical procedure or in immediate preparation for such procedure.

The term “alkyl” refers to a straight or branched hydrocarbon. For example, an alkyl group can have 1 to 6 carbon atoms (i.e, C₁-C₆ alkyl), 1 to 4 carbon atoms (i.e., C₁-C₄ alkyl), or 1 to 3 carbon atoms (i.e., C₁-C₃ alkyl).

The term “alkenyl” refers to a straight or branched hydrocarbon with at least one site of unsaturation, i.e. a carbon-carbon, sp² double bond. For example, an alkenyl group can have 2 to 6 carbon atoms (i.e., C₂-C₆ alkenyl) or 2 to 4 carbon atoms (i.e., C₂-C₄ alkenyl). Examples of suitable C₂-C₄ alkenyl groups include, but are not limited to, ethenyl or vinyl (—CH═CH₂), allyl (—CH₂CH═CH₂), but-1-enyl-CH═CH—CH₂—CH₃), but-2-enyl (CH₂—CH═CH—CH₃), but-3-enyl (—CH₂—CH₂—CH═CH).

### Methods of Use

Provided is a method of detecting nerves in a tissue or organ, the method comprising
a) administering an effective amount of a composition comprising a fluorophore as described herein to the tissue or organ to form a stained tissue or a stained organ; and
b) imaging the stained tissue or stained organ, thereby detecting nerves intraoperatively in the stained tissue or stained organ.

Provided is a method of detecting nerves intraoperatively in a subject undergoing surgery, the method comprising:
c) administering an effective amount of a composition comprising a fluorophore as described herein to the subject before or during surgery to form a stained tissue; and
d) imaging the stained tissue undergoing surgery in the subject, thereby detecting nerves intraoperatively in the subject undergoing surgery.

Also provided is a method of detecting nerves intraoperatively in a subject undergoing a prostatectomy surgery, the method comprising:
e) administering an effective amount of a composition comprising a fluorophore as described herein to the subject before or during the prostatectomy surgery to form a stained tissue; and
f) imaging the stained tissue undergoing surgery in the subject, thereby detecting nerves intraoperatively in the subject undergoing prostatectomy surgery.

In one embodiment is provided a method of detecting cavernous nerves intraoperatively in a subject undergoing a prostatectomy surgery, the method comprising:
g) administering an effective amount of a composition comprising a fluorophore as described herein to the subject before or during the prostatectomy surgery to form a stained tissue; and
h) imaging the stained tissue undergoing surgery in the subject, thereby detecting cavernous nerves intraoperatively in the subject undergoing prostatectomy surgery.

For each of the methods herein concerning a prostatectomy surgery or procedure, there is another embodiment in which the surgery or procedure is a radical prostatectomy.

For each of the methods above and herein, there is an embodiment in which the composition comprising a fluorophore is administered to the subject systemically.

For each of the methods above and herein, there is an embodiment in which the composition comprising a fluorophore is administered to the subject directly or topically, i.e. through direct administration or topical administration.

Within each of the methods herein, there is a further embodiment in which the administration of an effective amount of a composition comprising a fluorophore as described herein to the subject before or during the prostatectomy surgery to form a stained tissue can be completed in fifteen minutes or less. In a still further embodiment, the administration of an effective amount of a composition comprising a fluorophore as described herein to the subject before or during the prostatectomy surgery to form a stained tissue can be completed in ten minutes or less.

Also provided herein are methods of imaging nervous tissue tumors (neoplasms), including Gliomas, such as gliomatosis cerberi, Oligoastrocytomas, Choroid plexus papillomas, Ependymomas, Astrocytomas (Pilocytic astrocytomas and Glioblastoma multiforme), Dysembryoplastic neuroepithelial tumors, Oligodendrogliomas, Medulloblastomas, and Primitive neuroectodermal tumors; Neuroepitheliomatous tumors, such as Ganglioneuromas, Neuroblastomas, Atypical teratoid rhabdoid tumors, Retinoblastomas, and Esthesioneuroblastomas; and Nerve Sheath Tumors, such as Neurofibromas (Neurofibrosarcomas and Neurofibromatosis), Schwannomas, Neurinomas, Acoustic neuromas, and Neuromas.

Provided is a method of imaging a target area in a subject, the method comprising contacting the target area in the subject with a compound selected from those herein and detecting the compound in the target using fluorescence or near-infrared imaging.

Also provided is a method of imaging one or more nerves in a target area in a subject, the method comprising contacting the target area in the subject with a compound selected from those herein and detecting the compound in the target using fluorescence imaging.

Also provided is a method of imaging one or more nerves in a target area in a subject, the method comprising contacting the target area in the subject with a compound selected from those herein and detecting the compound in the target using near-infrared imaging.

Also provided is a method of minimizing nerve damage in a target area in a subject during a medical procedure, the method comprising the steps of:
a) contacting the target area in the subject with a compound selected from those herein;
b) detecting one or more nerves bound by the compound in the target area using fluorescence imaging; and
c) minimizing actions of the medical procedure that may damage one or more nerves detected.

The method above may be used to identify nerves and minimize damage to them that may be caused by a medical procedure, including traumatic, thermal, and radiological damage or that are caused by the application of therapeutic agents, anesthetics, or anesthesia in the target area.

In some embodiments, the medical procedure referenced in the method above is a surgical procedure. In other embodiments, the medical procedure is a biopsy procedure, a radiological procedure, or the application of anesthetic or anesthesia to the subject. In further embodiments, the medical procedure in the method above is the insertion or implantation of a medical device, including a medical pump, stent, pacemaker, port, artificial joints, valves, screws, pins, plates, rods, cosmetic implants, neurostimulators, and the like.

Also provided is the use of any compound disclosed herein in the preparation of a composition for use in imaging one or more nerves in a subject using from near-infrared imaging.

Nerve damage plagues surgical outcomes, significantly affecting post-surgical quality of life. Despite the practice of nerve sparing techniques for decades, intraoperative nerve identification and sparing remains difficult and success rates are strongly correlated with surgeon experience level and ability to master the technique (Walsh & Donker. The Journal of urology 128, 492-497 (1982); Ficarra et al. Eur Urol 62, 405-417 (2012); Damber & Khatami. Acta oncologica 44, 599-604 (2005)). Fluorescence-guided surgery (FGS) shows promise for enhanced visualization of specifically highlighted tissue, such as nerves and tumor tissue, intraoperatively. FGS using optical imaging technology is capable of real-time, wide field identification of targeted tissues with high sensitivity and specificity from tissue targeted fluorescent probes. See, for instance: Frangioni. Journal of clinical oncology: official journal of the American Society of Clinical Oncology 26, 4012-4021 (2008); Gibbs. Quantitative imaging in medicine and surgery 2, 177-187 (2012); Gioux et al. Molecular imaging 9, 237-255 (2010); Vahrmeijer et al. Nature reviews. Clinical oncology 10, 507-518 (2013); and Nguyen et al. Nature reviews. Cancer 13, 653-662 (2013). Operating in the near-infrared (NIR) optical window (650-900 nm wavelengths) where tissue chromophore absorbance, autofluorescence and scattering are minimal, FGS technologies have the ability to identify targeted tissues at millimeter to centimeter depths against a black background (Chance. Annals of the New York Academy of Sciences 838, 29-45 (1998); Gibbs. Quantitative imaging in medicine and surgery 2, 177-187 (2012)).

Several imaging systems have been developed for FGS applications. see, for instance: Lee et al. Plastic and reconstructive surgery 126, 1472-1481 (2010); Tummers et al. European journal of surgical oncology: the journal of the European Society of Surgical Oncology and the British Association of Surgical Oncology 40, 850-858 (2014); Troyan et al. Annals of surgical oncology 16, 2943-2952 (2009); Ashitate et al. Real-time simultaneous near-infrared fluorescence imaging of bile duct and arterial anatomy. The Journal of surgical research 176, 7-13 (2012); Verbeek et al. The Journal of urology 190, 574-579 (2013); Gibbs-Strauss et al. Molecular imaging 10, 91-101 (2011); Hirche et al. Surgical innovation 20, 516-523 (2013); Gotoh et al. Journal of surgical oncology 100, 75-79 (2009); and Kitagawa et al. Anticancer research 35, 6201-6205 (2015); Importantly, the da Vinci surgical robot, frequently used for robotic assisted radical prostatectomy (RP), can be equipped with an FDA approved fluorescence imaging channel.

Direct administration (also sometimes referred to as local administration) is an attractive alternative to systemic administration of fluorescent probes for minimizing potential toxicity and easing regulatory burdens for first in human clinical studies. By selectively labeling tissues within the surgical field, direct administration requires a significantly lower dose than systemic administration. A direct administration methodology has been developed that provides equivalent nerve signal to background (SBR) to systemic administration following a 15-minute staining protocol. Barth & Gibbs. Theranostics 7, 573-593 (2017). This methodology has been successfully applied to autonomic nerve models, which closely mimic the nerves surrounding the prostate. This method has additional benefits in the application to RP since nerve labeling via systemic administration during RP would generate high background from nerves in the prostate, which are not able to be spared, and renal fluorophore clearance, producing significant fluorescence signal in the urine within the adjacent bladder. Both of these extraneous fluorescence signals would diminish the ability to identify the cavernous nerves within the neurovascular bundle (NVB), which are responsible for continence and potency (Barth and Summer. Theranostics (2016). Tewari et al. BJU international 98, 314-323 (2006); Patel et al. Eur Urol 61, 571-576 (2012)). Perhaps most importantly, the direct administration methodology requires 16 times lower dose than systemic administration and when scaled to humans by body surface area the dose falls within the requirements for clinical translation under an exploratory investigational new drug (eIND) application to the FDA. Studies conducted under an eIND require minimal preclinical toxicity testing, since only a microdose (<100 μg) is administered to each patient, significantly reducing the cost of first-in-human studies.

While the direct administration methodology has provided high nerve specificity and SBR with a short staining protocol in preclinical rodent models (Barth & Gibbs. Theranostics 7, 573-593 (2017)), preliminary staining studies in large animal models generated significant background. To facilitate clinical translation, an improved formulation strategy that is FDA approved and facilitates increased application control for staining a variety of tissue surfaces, angles, and morphologies will be required.

Several classes of nerve specific fluorescence imaging probes have been studied preclinically for FGS. See, for instance: Gibbs-Strauss et al. Molecular imaging 10, 91-101 (2011); Wu et al. Journal of medicinal chemistry 51, 6682-6688 (2008); Wang et al. The journal of histochemistry and cytochemistry: official journal of the Histochemistry Society 58, 611-621 (2010); Gibbs et al. PloS one 8, e73493 (2013); Stankoff et al. Proceedings of the National Academy of Sciences of the United States of America 103, 9304-9309 (2006); Cotero et al. Molecular imaging and biology: MIB: the official publication of the Academy of Molecular Imaging 14, 708-717 (2012); Cotero et al. PloS one 10, e0130276 (2015); Bajaj et al. The journal of histochemistry and cytochemistry: official journal of the Histochemistry Society 61, 19-30 (2013); Gibbs-Strauss et al. Molecular imaging 9, 128-140 (2010); Meyers et al. The Journal of Neuroscience: the official journal of the Society for Neuroscience 23, 4054-4065 (2003); Wang et al. The Journal of neuroscience: the official journal of the Society for Neuroscience 31, 2382-2390 (2011); Park et al. Theranostics 4, 823-833 (2014). Of these, oxazine fluorophores (e.g., Oxazine 4) have demonstrated the most promise for clinical translation, with high nerve specificity following both direct and systemic administration. (3-(diethyl-14-azaneylidene)-N-ethyl-8-methyl-3H-phenoxazin-7-amine) is a particularly promising compound and was chosen as the lead compound for advancement to clinical studies. Although Comparative Example No. 1 has been shown to demonstrate high nerve specificity and adequate fluorescence signal for real time imaging, previous studies have been conducted utilizing a co-solvent formulation as a vehicle for intravenous injection (Gibbs-Strauss et al. Molecular imaging 10, 91-101 (2011); Barth & Gibbs. Theranostics 7, 573-593 (2017)). The co-solvent formulation is only stable at room temperature for <30 minutes, cannot solubilize concentrations above 5 mg/mL, and requires the use of dimethyl sulfoxide and Kolliphor EL as solubilizing agents, which hampers clinical translation due to vehicle induced toxicity issues. Additionally, the co-solvent formulation is liquid based and thus not ideal for staining angled or vertical tissue surfaces. Therefore, a clinically viable formulation with FDA approval was needed for direct administration and intravenous injection of nerve-specific fluorescence for FGS.

Formulations comprising one or more of the compounds disclosed herein can be used to image nerves or nerve tissue. In particular embodiments, the formulations of the disclosure can be used to image nerves or nerve tissue in a subject. In particular embodiments, images of nerves can be obtained intraoperatively during FGS. In particular embodiments, the visualization of nerves during FGS allows surgery to be performed on tissue of interest while sparing nerves so as to reduce incidence of nerve injury during surgery. The area where surgery is performed or nearby regions can be surgically exposed. Surgery can be performed on organs, which include tissues such as nerve tissue, muscle tissue, and adipose tissue. The surgery can be laparoscopic, which is minimally invasive and includes the use of a thin, tubular device (laparoscope) that is inserted through a keyhole incision into a part of a subject's body, such as the abdomen or pelvis. The surgery can be assisted by a robot. Robot-assisted surgery can offer more precision, flexibility, and control, and is often associated with minimally invasive surgery.

In particular embodiments, the fluorophore concentration in a formulation that is directly applied to nerve tissue includes a concentration range of 40 to 300 μg/mL. In particular embodiments, the fluorophore concentration in a formulation for direct application includes 40 μg/mL, 50 μg/mL, 60 μg/mL, 70 g/mL, 80 μg/mL, 90 μg/mL, 100 μg/mL, 110 μg/mL, 120 g/mL, 130 μg/mL, 140 μg/mL, 150 μg/mL, 160 μg/mL, 170 μg/mL, 180 μg/mL, 190 μg/mL, and 200 μg/mL. In particular embodiments, the fluorophore concentration in a formulation for direct application is 50 μg/mL. In particular embodiments, the fluorophore concentration in a formulation for direct application is 200 μg/mL.

A formulation of the disclosure can be systemically applied to a subject for imaging of nerves. In particular embodiments, systemic application of a formulation includes intravenous injection of the formulation into a subject.

A formulation that is directly applied to a tissue can be allowed to penetrate the tissue for a given amount of time after direct application. In particular embodiments, the formulation can be allowed to penetrate the tissue for 30 seconds to 15 minutes, for 1 to 10 minutes, for 1 to 5 minutes, for 1 minute, for 2 minutes, for 3 minutes, for 4 minutes, or for 5 minutes. In particular embodiments, the formulation can be allowed to penetrate the tissue for 1 to 2 minutes. A formulation that is systemically applied to a subject can be administered a sufficient time before imaging such that the formulation can reach the area to be imaged and is present in such area at the time of imaging. In particular embodiments, a formulation that is systemically applied to a subject can be administered a sufficient time prior to imaging to allow uptake of the formulation by tissue in the subject. In particular embodiments, the formulation may be administered up to or less than 30 minutes, 1 hour, 2 hours, 3 hours, 4 hours, 5 hours, 6 hours, 7 hours, or 8 hours before imaging. The amount of time required may depend on the nerve imaging application and the administration site. In particular embodiments, the formulation is administered no more than 30 minutes, 1 hour, 2 hours, 3 hours, or 4 hours before imaging. In particular embodiments, the formulation is administered no more than 2 hours before imaging.

Tissue stained by a formulation including a fluorophore by direct application can be washed with buffer prior to imaging of the stained tissue. Washing of tissue stained by a formulation including a fluorophore can include flushing the tissue with an appropriate buffer and removing the buffer. In particular embodiments, the stained tissue can be washed 1 to 18 times, 1 to 10 times, 1 to 6 times, 1 time, 2 times, 3 times, 4 times, 5 times, or 6 times, with wash buffer. In particular embodiments, the stained tissue can be washed 6 times. In particular embodiments, the wash buffer is phosphate-buffered saline (PBS). In particular embodiments, washing the stained tissue removes unbound fluorophore. In particular embodiments, washing the stained tissue increases the nerve signal intensity and/or the signal to background ratio (SBR) as compared to no washing of the stained tissue. In particular embodiments, washing the stained tissue resolubilizes the fluorophore and allows for further diffusion of the fluorophore into the nerve tissue.

Imaging a tissue stained by a formulation including a fluorophore includes applying light to tissue that has been stained with a formulation of the disclosure. The light can be at a wavelength sufficient to excite the fluorophore in the formulation to fluoresce. In particular embodiments, light to excite the fluorophore is at a wavelength in the near infrared spectra. In particular embodiments, the fluorophore of a formulation emits at a wavelength in the near infrared spectra. In particular embodiments, the near infrared spectra includes a wavelength of 700 to 900 nm.

Imaging a tissue stained by a formulation including a fluorophore includes obtaining fluorescence images of the stained tissue by optical imaging systems such as ones described in the Examples.

In particular embodiments, imaging a tissue includes observing fluorescence images of the stained tissue. The fluorescence images can include still images (whether printed or on screen), or real-time images on a video monitor. In particular embodiments, the individual images of nerves obtained by staining of the nerves with the present formulations can be used for diagnostic purposes and for documentation of nerve location. By observing the fluorescence images the surgical team can determine the absence or presence of a nerve in the image. The surgical team can thus use information about the presence/absence or location of one or more nerves to determine how they will perform the surgical procedure. For example, based on information obtained through the disclosed methods, the surgical team may decide to perform a surgical cut at a point in the tissue where they are less likely to inadvertently cut or surgically contact a particular nerve based on the perceived absence of a nerve in an area of the tissue.

The information obtained from the obtained image can aid in grafting the ends of the nerves if they are transected. In the event of transection, nerve grafts can be applied directly to the ends to facilitate sprouting of regenerative neural fibers. In this case, the light visible from the fluorescence of the ends of transected nerves provides a target to guide the anastomosis of the nerves by the nerve graft.

Formulations of the present disclosure to detect nerve tissue can also be provided as kits. Kits for detecting nerve tissue can include, in different containers: (i) a water-based formulation comprising a fluorophore, and (ii) one or more wash buffers. Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding: directly applying the formulations to a tissue; washing to remove excess formulation; systemically administering the formulations to a subject; applying light for visualization of the fluorophores; capturing fluorescent images of the tissue; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary laboratory and/or medical supplies needed to use the kit effectively, such as syringes, ampules, tubing, gloves, tubes, buffers, and the like. Variations in contents of any of the kits described herein can be made.

### General

All reagents were purchased from Sigma Aldrich, Fisher Scientific, or TCI. Unless otherwise indicated, all commercially available starting materials were used directly without further purification. Analytical TLC was performed on Millipore ready-to-use plates with silica gel 60 (F254, 32-63 μm). Purification was performed on a Biotage Isolera Flash System using pre-packed silica gel cartridges or on a reverse phase preparative HPLC (Agilent 1250 Infinity HPLC).

### LCMS Characterization

Mass-to-charge ratio and purity of the Oxazine compounds were characterized on an Agilent 6244 time-of-flight LCMS with diode array detector VL+. Sample (10 uL) was injected into a C₁₈ column (Poroshell 120, 4.6×50 mm, 2.7 micron), and eluted with a solvent system of A (H₂O, 0.1% FA) and B (MeCN, 0.1% FA) at 0.4 mL/min, from A/B=90/10 to 5/95 over 10 min, maintained at A/B=5/95 for additional 5 min. Ions were detected in positive ion mode by setting the capillary voltage at 4 kV and gas temperature at 350° C.

### UV-Vis Absorption and Fluorescence Spectroscopy

UV-Vis and fluorescence spectra were collected on a SpectraMax M5 spectrometer with a Microplate reader (Molecular Devices, Sunnyvale, CA). All absorbance spectra were reference corrected. Extinction coefficient was calculated from Beer's Law plots of absorbance versus concentration. Relative quantum yields are reported using HITCI as reference. Excitation emission matrices (EEMs) were collected on a Cary Eclipse fluorescence spectrophotometer (Agilent Technologies), using 5-nm step size. The band pass for excitation and emission was 10 nm.

### Water Solubility Measurements.

Each screening candidate was dissolved in a 1 mL mixture of chloroform and methanol (equal volume) with final stock concentrations ranging from 10 to 50 mM. The solvent was then removed in vacuo before 200 μL of DI water was added. The test sample was then vortexed before sonicated in an ultrasonic bath for 30 minutes. The undissolved pellet was removed by centrifugation at 13,000 rpm for 5 minutes. The supernatant was sampled and diluted with water before measured for absorbance using a SpectraMax M5 spectrometer with a Microplate reader (Molecular Devices, Sunnyvale, CA). The water solubility of each screening candidate was then calculated using Beer's Law plots of absorbance versus concentration. The water solubility concentration unit (mM) of each sample was then converted and reported as mg/mL.

### Experimental Log D Measurements.

Each screening candidate was dissolved in DMSO at a concentration of 10 mM. The stock solution was sampled (2 μL) and added to a 1 mL mixture of 1-octanol and PBS buffer (equal volume). The solution was then vortexed for 30 mins at room temperature before centrifuged at 13,000 rpm for 5 minutes. The PBS buffer and 1-octanol layers were separated and measured for absorbance using a SpectraMax M5 spectrometer with a Microplate reader (Molecular Devices, Sunnyvale, CA). Sample concentration in each phase was then calculated using Beer's Law plots of absorbance versus concentration. The experimental Log D value for each screening candidates was calculated using the equation below.

$$Log D = Log ( \frac{Sample concentration in PBS buffer}{Sample concetration in 1 - octanol} )$$

### Nerve-Specificity Screening Using Direct/Topical Administration

Each compound was screened for its tissue-specificity using a previously published direct/topical administration strategy in murine brachial plexus and sciatic nerves. Each compound from the nerve dye library was formulated in phosphate buffered saline solution at 125 μM. 100 μL of the formulated Oxazine were incubated on the exposed brachial plexus or sciatic nerve for 5 minutes. The fluorophore containing solution was removed and the area was irrigated with saline 18 times to remove any unbound fluorophore. Co-registered fluorescence and color images were collected of each stained area 30 minutes after Oxazine direct/topical administration using a custom-built macroscopic imaging system with 620/60 nm excitation and 700/75 nm bandpass emission filters. Custom written MatLab code was used to analyze the tissue specific fluorescence where regions of interest were selected on the nerve, muscle and adipose tissue using the white light images. These regions of interest were then analyzed on the co-registered matched fluorescence images permitting assessment of the nerve to muscle and nerve to adipose ratios.

Nerve-Specificity Screening using Systemic Administration

Each compound was screened for its tissue-specificity using a previously published systemic administration strategy in murine brachial plexus and sciatic nerves. Each compound from the Oxazine library was formulated in phosphate buffered saline solution at 2 mM. 100 μL of the formulated Oxazine were administered intravenously 2 hours before exposing the brachial plexus and sciatic nerves. Co-registered fluorescence and color images were collected of each nerve site using a custom-built macroscopic imaging system with 620/60 nm excitation and 700/75 nm bandpass emission filters. Custom written MatLab code was used to analyze the tissue specific fluorescence where regions of interest were selected on the nerve, muscle and adipose tissue using the white light images. These regions of interest were then analyzed on the co-registered matched fluorescence images permitting assessment of the nerve to muscle and nerve to adipose ratios in blinded manner.

### Chemical Synthesis

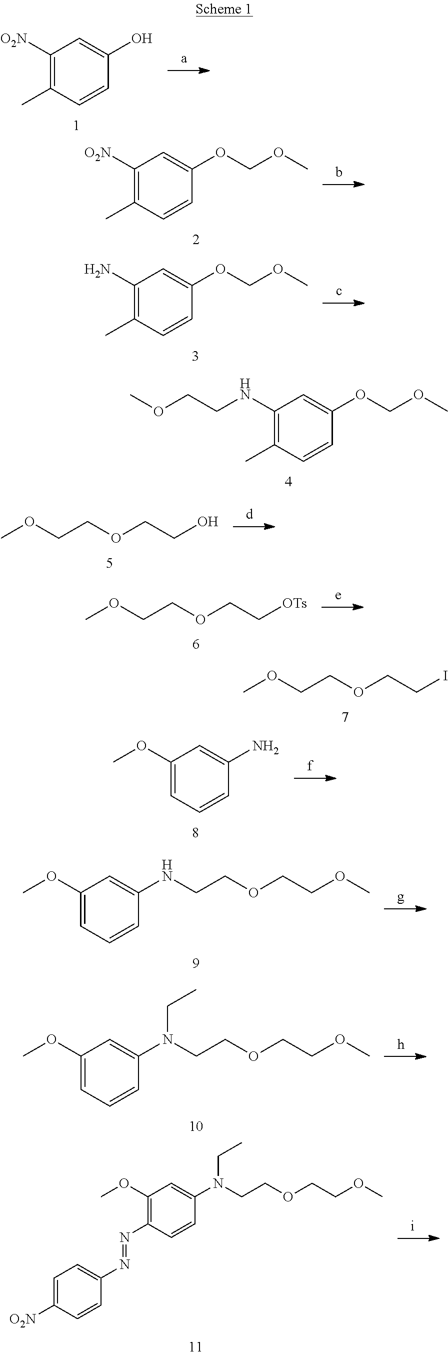

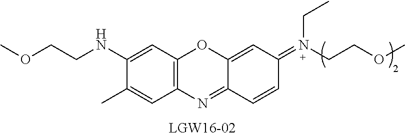

Synthetic route to LGW16-02. Reagents and conditions: a) NaH, MOMCI, DMF, 0° C. to rt; b) Fe, NH₄Cl, 90% EtOH, 110° C.; c) NaH, 2-Bromoethyl methyl ether, DMF, 0 to 80° C.; d) TsCl, NaOH, THF/H₂O, 0° C. to rt; e) NaI, Acetone, rt; f) compound 7, K₂CO₃, MeCN, 80° C.; g) Etl, Na₂CO₃, MeCN, 80° C.; h) I) 2M HCl, p-nitrobenzenediazonium tetrafluoroborate, 0° C.; II) K₂CO₃, 0° C.; i) compound 4, HClO₄, 90% i-PrOH, 80° C.

4-(methoxymethoxy)-1-methyl-2-nitrobenzene (2): A solution of compound 1 (20.0 g, 131 mmol) in anhydrous DMF (25 mL) was stirred in an ice bath under N₂ for 30 mins. NaH (60%, 6.27 g, 157 mmol) was added to the solution above portion wise. The temperature of the solution was maintained below 5° C. and stirred for 1 h before MOMCI (10.91 mL, 144 mmol) was added dropwise. The resulting reaction mixture was stirred for an additional 1 h in the ice bath, and then at rt for 3 h. The reaction was then chilled in an ice bath and quenched with 1M aqueous HCl solution (100 mL). The aqueous solution was extracted with EtOAc (3×100 mL), and the combined organic layers were washed with 1M aqueous HCl (100 mL), 5% LiCl (100 mL), and brine (100 mL) before dried over anhydrous Na₂SO₄ and concentrated in vacuo. The residue (clear liquid, quantitative) was used for the next step without further purification.

5-(methoxymethoxy)-2-methylaniline (3): 1 L round bottom flask was charged with compound 2 (25.0 g, 127 mmol), ion dust (38.9 g, 697 mmol), NH₄Cl (4.75 g, 88.8 mmol) and a magnetic stir bar. To the flask was added a solution of EtOH/H₂O (9/1, 500 mL). The resulting suspension was stirred at 110° C. for 5 h, before cooled to rt. The solid was removed via vacuum filtration and the solvent was removed under reduced pressure. The residue was then resuspended in saturated NaHCO₃ solution (100 mL), and extracted with CHCl₃ (3×100 mL). The combined organic layers were then dried over anhydrous Na₂SO₄ and concentrated in vacuo. Purification by flash column chromatography using a gradient of EtOAc (10-40%) in hexanes as eluent afforded compound 3 (18.9 g, 89%) as a clear oil.

N-(2-methoxyethyl)-5-(methoxymethoxy)-2-methylaniline (4): A solution of compound 3 (2.0 g, 12.0 mmol) in anhydrous DMF (10 mL) was stirred in an ice bath under N₂ for 30 mins. NaH (60%, 574 mg, 14.4 mmol) was added to the solution above portion wise. The temperature of the solution was maintained below 5° C. and stirred for 1 h before a DMF (10 mL) solution of 2-Bromoethyl methyl ether (1.35 mL, 14.4 mmol) was added dropwise. The resulting reaction mixture was stirred at RT for 1 h, and then at 80° C. for 5 h. The reaction was then cooled to rt and quenched with DI water. The resulting mixture was diluted with saturated NaHCO₃ solution (50 mL) and extracted with EtOAc (3×50 mL). The combined organic layers were washed with 5% LiCl (50 mL), and brine (50 mL) before dried over anhydrous Na₂SO₄ and concentrated in vacuo. Purification by flash column chromatography using a gradient of EtOAc (5-15%) in hexanes as eluent afforded compound 4 (431 mg, 16%) as a clear oil.

2-(2-methoxyethoxy)ethyl 4-methylbenzenesulfonate (6): To a THF solution (25 mL) of diethylene glycol methyl ether (5 g, 41.6 mmol) was added NaOH (20%, 25 mL). The resulting solution was chilled in an ice bath before TsCl (9.52 g, 49.9 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

1-iodo-2-(2-methoxyethoxy) ethane (7): To an acetone (60 mL) solution of compound 6 (6.2 g, 22.6 mmol) was added NaI (5.08 g, 33.9 mmol) in one portion. The reaction mixture was covered from light and stirred at rt overnight. The solid was removed via vacuum filtration through Celite and the filtrate was concentrated using a rotary evaporator. The residue was diluted with EtOAc (100 mL) and washed with DI water (100 mL). The aqueous phase was then extracted with EtOAc (3×100 mL). The combined organic layers were washed with 1% NaHSO₃ (50 mL), brine and dried over anhydrous Na₂SO₄, then concentrated in vacuo. Purification by flash column chromatography using a gradient of EtOAc (2-20%) in hexanes as eluent afforded compound 7 (3.82 g, 73%) as a yellow oil.

3-methoxy-N-(2-(2-methoxyethoxy)ethyl) aniline (9): Compound 8 (1.0 g, 8.12 mmol), compound 7 (1.2 g, 9.74 mmol), and K₂CO₃ (1.68 g, 12.2 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel, using EtOAc (50-100%) in hexanes as eluent to give compound 9 (1.24 g, 68%).

N-ethyl-3-methoxy-N-(2-(2-methoxyethoxy)ethyl) aniline (10): Compound 9 (0.50 g, 2.22 mmol), Etl (0.69 g, 4.44 mmol), and Na₂CO₃ (0.35 g, 3.33 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel, using a gradient of EtOAc (5-20%) in hexanes as eluent to give compound 10 (449 mg, 80%) as a light-brown oil.

(E)-N-ethyl-3-methoxy-N-(2-(2-(2-methoxyethoxy) ethoxy)ethyl)-4-((4-nitrophenyl)diazenyl) aniline (11): Compound 10 (300 mg, 1.18 mmol) was dissolved in MeOH (2 mL). The solution was chilled in an ice bath, then was treated with HCl (2 M, 20 mL). After 15 mins, p-nitrobenzenediazonium tetrafluoroborate (295 mg, 1.24 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

(E)-N-ethyl-2-(2-methoxyethoxy)-N-(7-((2-methoxyethyl)amino)-8-methyl-3H-phenoxazin-3-ylidene) ethan-1-aminium (LGW16-02): A 2-dram vial was charged with compound 4 (50 mg, 0.22 mmol), compound 11 (94 mg, 0.23 mmol), and a magnetic stir bar. To the vial was added a solution of i-PrOH/H₂O (9:1, 4 mL) and HClO₄ (70%, 40 μL). The resulting solution was stirred at 90° C. for 4 h to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-02 (60 mg, 65%).

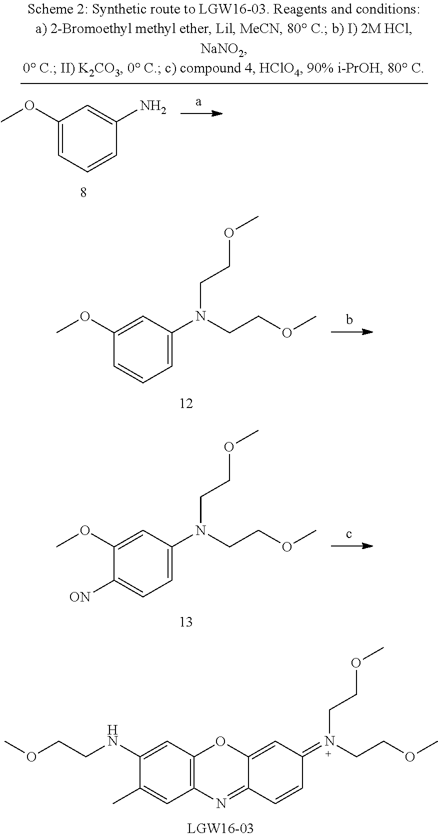

3-methoxy-N,N-bis(2-methoxyethyl) aniline (12): Compound 8 (1.0 g, 8.12 mmol), 2-bromoethyl methyl ether (2.31 mL, 24.4 mmol), Lil (761 mg, 5.68 mmol), and K₂CO₃ (2.24 g, 16.2 mmol) were suspended in anhydrous MeCN (20 mL) under N₂. The reaction mixture was then heated to 80° C. and stirred for 12 h prior to the addition of 2-bromoethyl methyl ether (0.76 mL, 8.12 mmol). The reaction mixture was stirred for an additional 12 h before cooled to rt and diluted with DCM (50 mL). The solid was removed via vacuum filtration through Celite. The solvent was removed using a rotary evaporator and the residue was purified by flash column chromatography using a gradient of DCM (50-100%) in hexanes as eluent afforded compound 12 (1.62 g, 83%) as a clear oil.

3-methoxy-N,N-bis(2-methoxyethyl)-4-nitrosoaniline (13): Compound 12 (1.2 g, 5.01 mmol) was dissolved in an ice-cold 2 M HCl solution (15 mL). To this solution was added NaNO₂ (381 mg, 5.52 mmol) portion-wise over 0.5 h while the temperature of the solution was maintained below 5° C., such that no brown NOx vapors were observed. The reaction mixture was stirred for an additional 2 h in the ice bath. The solution was carefully basified with solid K₂CO₃ until the pH value of the solution had risen above 8. The aqueous solution was then extracted with DCM (3×50 mL), and the combined organic layers were washed with brine (50 mL) before dried over anhydrous Na₂SO₄ and concentrated in vacuo. The title compound was obtained (1.03 g, 77%) as a dark green viscous oil, which was used for the next step without further purification.

2-methoxy-N-(2-methoxyethyl)-N-(7-((2-methoxyethyl)amino)-8-methyl-3H-phenoxazin-3-ylidene) ethan-1-aminium (LGW16-03): A 2-dram vial was charged with compound 4 (50 mg, 0.22 mmol), compound 13 (63 mg, 0.23 mmol), and a magnetic stir bar. To the vial was added a solution of i-PrOH/H₂O (9:1, 5 mL) and HClO₄ (70%, 50 μL). The resulting solution was stirred at 90° C. for 3 h to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-03 (63 mg, 71%).

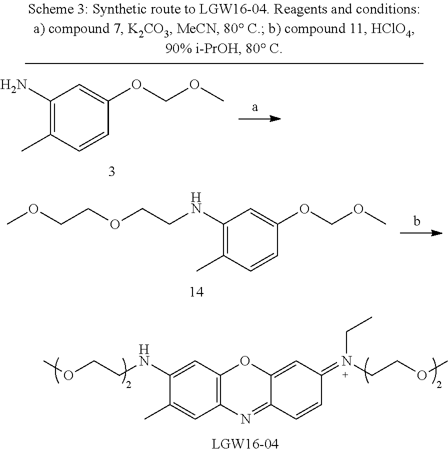

N-(2-(2-methoxyethoxy)ethyl)-5-(methoxymethoxy)-2-methylaniline (14): Compound 3 (1.0 g, 5.98 mmol), compound 7 (1.65 g, 7.18 mmol), and K₂CO₃ (1.24 g, 8.97 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel, using EtOAc (10-30%) in hexanes as eluent to give compound 14 (1.01 g, 63%) as a light-orange oil.

(E)-N-ethyl-2-(2-methoxyethoxy)-N-(7-((2-(2-methoxyethoxy)ethyl) amino)-8-methyl-3H-phenoxazin-3-ylidene) ethan-1-aminium (LGW16-04): A 2-dram vial was charged with compound 14 (50 mg, 0.19 mol), compound 11 (78 mg, 0.20 mmol), and a magnetic stir bar. To the vial was added a solution of i-PrOH/H₂O (9:1, 4 mL) and HClO₄ (70%, 40 μL). The resulting solution was stirred at 90° C. overnight to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-04 (28 mg, 32%).

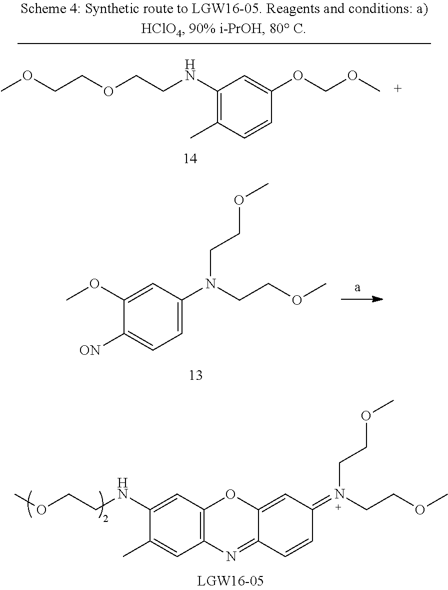

2-methoxy-N-(7-((2-(2-methoxyethoxy)ethyl) amino)-8-methyl-3H-phenoxazin-3-ylidene)-N-(2-methoxyethyl) ethan-1-aminium (LGW16-05): A 2-dram vial was charged with compound 14 (50 mg, 0.19 mmol), compound 13 (52 mg, 0.20 mmol), and a magnetic stir bar. To the vial was added a solution of i-PrOH/H₂O (9:1, 4 mL) and HClO₄ (70%, 40 μL). The resulting solution was stirred at 90° C. for 3 h to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-05 (49 mg, 59%).

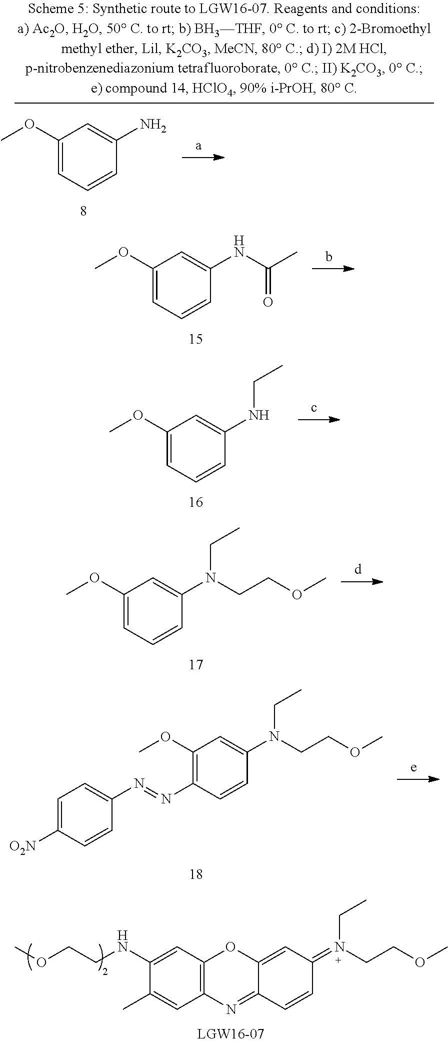

N-(3-methoxyphenyl) acetamide (15): Compound 8 (2 g, 16.2 mmol) was suspended in 50 mL DI water, to which Acetic anhydride (4.61 mL, 48.7 mmol) was added slowly. The reaction mixture was placed in an ultrasonication bath for 1 min, then was stirred in a water bath at 50° C. for 10 min. The resulting solution was stirred overnight at rt. The reaction mixture was chilled in an ice bath and carefully neutralized with NaOH (10%) aqueous solution. The aqueous solution was then extracted with DCM (3×50 mL), and the combined organic layers were washed with brine, dried over anhydrous Na₂SO₄, then concentrated in vacuo. The product was left in the funnel and air dried overnight to afford compound 15 (2.22 g, 83%) as a light brown oil, which was solidified upon cooling in the fridge, compound 15 was used for the next step without further purification.

N-ethyl-3-methoxyaniline (16): A solution of 15 (2.0 g, 12.2 mmol) in anhydrous THF (35 mL) was stirred in an ice bath under N₂ for 30 mins. Borane tetrahydrofuran complex solution (1 M, 35 mL) was added to the solution above using a syringe pump over 30 mins, while maintaining the temperature of the solution below 5° C. The resulting reaction mixture was left in the ice bath and slowly warmed to rt. After 24 h, the solution was placed in an ice bath again, and excess borane reagent was destroyed by carefully adding MeOH until no gas evolved. The solvent was evaporated under reduced pressure, and the residue was purified by flash column chromatography with silica gel, using a gradient of EtOAc (5-20%) in hexanes as eluent to obtain compound 16 (1.39 g, 75%) as a brown oil.

N-ethyl-3-methoxy-N-(2-methoxyethyl) aniline (17): Compound 16 (1.0 g, 6.61 mmol), 2-Bromoethyl methyl ether (1.38 g, 9.92 mmol), Lil (620 mg, 4.63 mmol), and K₂CO₃ (1.37 g, 9.92 mmol) were suspended in anhydrous MeCN (20 mL) under N₂. The reaction mixture was then heated to 80° C. and stirred for 24 h before a second addition of 2-Bromoethyl methyl ether (1.38 g, 9.92 mmol). The reaction mixture was stirred for another 48 h before diluted with DCM (40 mL). The solid was removed via vacuum filtration through Celite. The solvent was removed using a rotary evaporator and the residue was purified by flash column chromatography with silica gel, using a gradient of DCM (10-40%) in hexanes as eluent to give compound 17 (1.17 g, 85%) as a clear oil.

(E)-N-ethyl-3-methoxy-N-(2-methoxyethyl)-4-((4-nitrophenyl)diazenyl) aniline (18): Compound 17 (300 mg, 1.43 mmol) was dissolved in MeOH (2 mL). The solution was chilled in an ice bath, then was treated with HCl (2 M, 20 mL). After 15 mins, p-nitrobenzenediazonium tetrafluoroborate (374 mg, 1.58 mmol) was added to the solution in 3 portions over 15 mins, then stirred at 0° C. for an additional 2 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

(E)-N-ethyl-2-methoxy-N-(7-((2-(2-methoxyethoxy)ethyl) amino)-8-methyl-3H-phenoxazin-3-ylidene) ethan-1-aminium (LGW16-07): A 2-dram vial was charged with compound 14 (50 mg, 0.19 mmol), compound 18 (70 mg, 0.20 mmol), and a magnetic stir bar. To the vial was added a solution of i-PrOH/H₂O (9:1, 4 mL) and HClO₄ (70%, 40 μL). The resulting solution was stirred at 90° C. for 4 h to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-07 (29 mg, 37%).

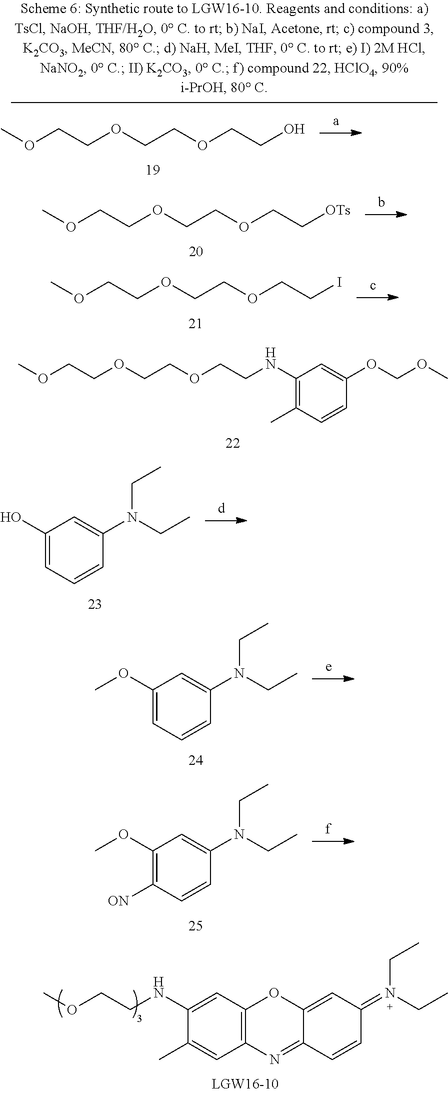

2-(2-(2-methoxyethoxy) ethoxy)ethyl 4-methylbenzenesulfonate (20): To a THF solution (25 mL) of Triethylene glycol monomethyl ether (5 g, 30.5 mmol) was added NaOH (20%, 25 mL). The resulting solution was chilled in an ice bath before TsCl (6.97 g, 36.5 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

1-iodo-2-(2-(2-methoxyethoxy) ethoxy) ethane (21): To an acetone (20 mL) solution of compound 20 (1.5 g, 4.71 mmol) was added NaI (1.06 g, 7.07 mmol) in one portion. The reaction mixture was covered from light and stirred at rt overnight. The solid was removed via vacuum filtration through Celite and the filtrate was concentrated using a rotary evaporator. The residue was diluted with EtOAc (50 mL) and washed with DI water (50 mL). The aqueous phase was then extracted with EtOAc (3×50 mL). The combined organic layers were washed with 1% NaHSO₃ (50 mL), brine, and dried over anhydrous Na₂SO₄, then concentrated in vacuo. Purification by flash column chromatography using a gradient of EtOAc (5-30%) in hexanes as eluent afforded compound 21 (0.92 g, 72%) as a yellow oil.

N-(2-(2-(2-methoxyethoxy) ethoxy)ethyl)-5-(methoxymethoxy)-2-methylaniline (22): Compound 3 (1.0 g, 5.98 mmol), compound 21 (1.97 g, 7.18 mmol), and K₂CO₃ (1.24 g, 8.97 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel, using EtOAc (15-50%) in hexanes as eluent to give compound 22 (1.20 g, 64%) as a brown oil.

N,N-diethyl-3-methoxyaniline (24): Compound 23 (5.0 g, 30.2 mmol) was dissolved in anhydrous THF (50 mL) under N₂ and chilled in an ice bath for 30 mins. NaH (60%, 3.63 g, 90.8 mmol) was added to the solution in 3 portions over 10 mins while the temperature was kept below 5° C. After 10 mins, Mel (7.54 mL, 121 mmol) was added into the reaction mixture in one portion. The resulting suspension was slowly warmed up to rt and stirred overnight. Upon completion, DI water was added to the reaction mixture to destroy excess NaH. Organic solvent was removed under reduced pressure and the residue was extracted with DCM (3×100 mL). The combined organic layers were rinsed with brine, dried over anhydrous Na₂SO₄, and the solvent was removed using a rotary evaporator. The residue was purified by flash column chromatography with silica gel, using a gradient of DCM (50-100%) in hexanes as eluent to give compound 24 (4.7 g, 88%) as a clear oil.

N,N-diethyl-3-methoxy-4-nitrosoaniline (25): Compound 24 (1.08 g, 6.02 mmol) was dissolved in an ice-cold 2 M HCl solution (15 mL). To the solution above, NaNO₂ (457 mg, 6.63 mmol) was added portion wise over 1 h while maintaining the temperature of the solution below 5° C., such that no brown NOx vapors were observed. The reaction mixture was stirred for an additional 2 h. The solution was carefully basified with solid K₂CO₃ until pH value of the solution rose above 8. After which, the precipitate was filtered through a Büchner funnel and washed with small portions of DI water. The product was left in the funnel and air dried overnight to afford compound 25 (1.05 g, 84%) as a green solid, which was used for the next step without further purification.

N-ethyl-N-(7-((2-(2-(2-methoxyethoxy) ethoxy)ethyl) amino)-8-methyl-3H-phenoxazin-3-ylidene) ethanaminium (LGW16-10): A 2-dram vial was charged with compound 22 (50 mg, 0.16 mmol), compound 25 (35 mg, 0.17 mmol), and a magnetic stir bar. To the vial was added a solution of i-PrOH/H₂O (9:1, 3 mL) and HClO₄ (70%, 30 μL). The resulting solution was stirred at 90° C. for 4 h to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-10 (14 mg, 21%).

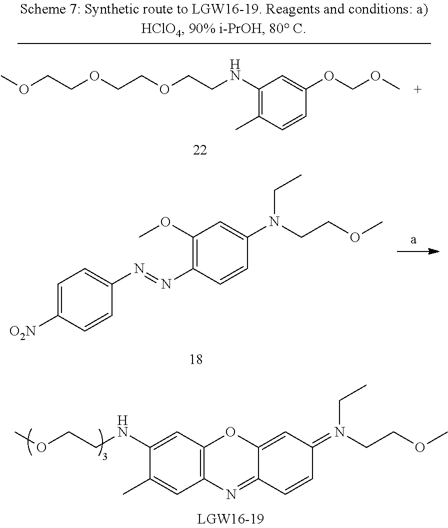

(E)-N-ethyl-2-methoxy-N-(7-((2-(2-(2-methoxyethoxy) ethoxy)ethyl) amino)-8-methyl-3H-phenoxazin-3-ylidene) ethan-1-aminium (LGW16-19): A 2-dram vial was charged with compound 22 (50 mg, 0.16 mmol), compound 18 (60 mg, 0.17 mmol), and a magnetic stir bar. To the vial was added a solution of i-PrOH/H₂O (9:1, 3 mL) and HClO₄ (70%, 30 μL). The resulting solution was stirred at 90° C. for 4 h to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-19 (41 mg, 57%).

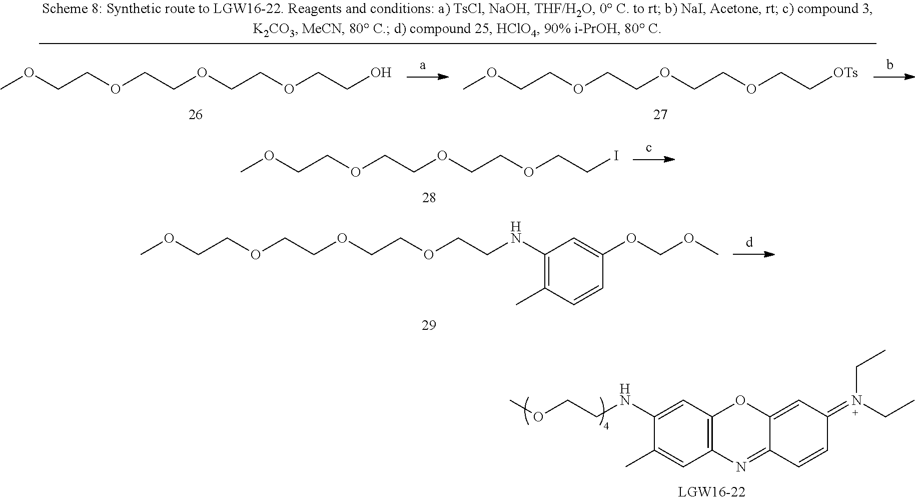

2,5,8,11-tetraoxatridecan-13-yl 4-methylbenzenesulfonate (27): To a THF solution (25 mL) of Tetraethyleneglycol monomethyl ether 36 (5 g, 24.0 mmol) was added NaOH (20%, 25 mL). The resulting solution was chilled in an ice bath before TsCl (6.01 g, 28.8 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

13-iodo-2,5,8,11-tetraoxatridecane (28): To an acetone (15 mL) solution of compound 27 (1.3 g, 3.59 mmol) was added NaI (0.807 g, 5.38 mmol) in one portion. The reaction mixture was covered from light and stirred at rt overnight. The solid was removed via vacuum filtration through Celite and the filtrate was concentrated using a rotary evaporator. The residue was diluted with EtOAc (25 mL) and washed with DI water (25 mL). The aqueous phase was then extracted with EtOAc (3×25 mL). The combined organic layers were washed with 1% NaHSO₃ (25 mL), brine, and dried over anhydrous Na₂SO₄, then concentrated in vacuo. Purification by flash column chromatography using a gradient of EtOAc (20-50%) in hexanes as eluent afforded compound 28 (0.77 g, 68%) as a yellow oil.

N-(5-(methoxymethoxy)-2-methylphenyl)-2,5,8,11-tetraoxatridecan-13-amine (29): Compound 3 (0.35 g, 2.09 mmol), compound 28 (0.80 g, 2.51 mmol), and K₂CO₃ (0.43 g, 3.14 mmol) were suspended in anhydrous MeCN (10 mL) under N₂. The reaction mixture was then heated to 80° C. and stirred for 24 h before diluted with DCM (20 mL). The solid was removed via vacuum filtration through Celite. The solvent was removed using a rotary evaporator and the residue was purified by flash column chromatography with silica gel, using EtOAc (50-100%) in hexanes as eluent to give compound 29 (0.47 g, 63%) as a red oil.

N-(7-((2,5,8,11-tetraoxatridecan-13-yl)amino)-8-methyl-3H-phenoxazin-3-ylidene)-N-ethylethanaminium (LGW16-22): A 2-dram vial was charged with compound 22 (100 mg, 0.28 mmol), compound 25 (61 mg, 0.294 mmol), and a magnetic stir bar. To the vial was added a solution of i-PrOH/H₂O (9:1, 5 mL) and HClO₄ (70%, 50 μL). The resulting solution was stirred at 90° C. overnight to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-22 (18 mg, 14%).

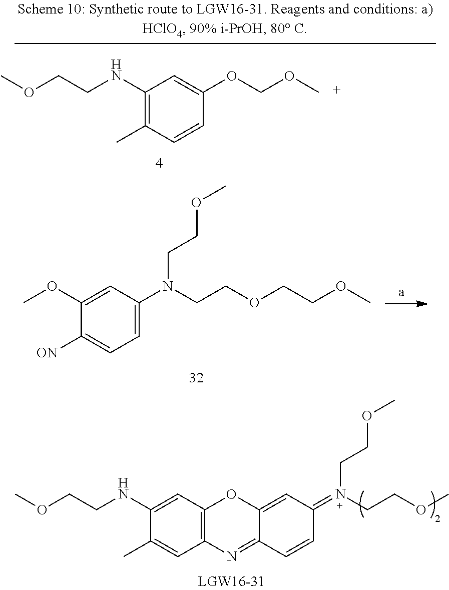

(E)-2-methoxy-N-(2-(2-methoxyethoxy)ethyl)-N-(7-((2-methoxyethyl)amino)-8-methyl-3H-phenoxazin-3-ylidene) ethan-1-aminium (LGW16-31): A 2-dram vial was charged with compound 4 (50 mg, 0.22 mmol), compound 32 (73 mg, 0.23 mmol), and a magnetic stir bar. To the vial was added a solution of i-PrOH/H₂O (9:1, 3 mL) and HClO₄ (70%, 30 L). The resulting solution was stirred at 90° C. for 4 h to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-31 (48 mg, 49%).

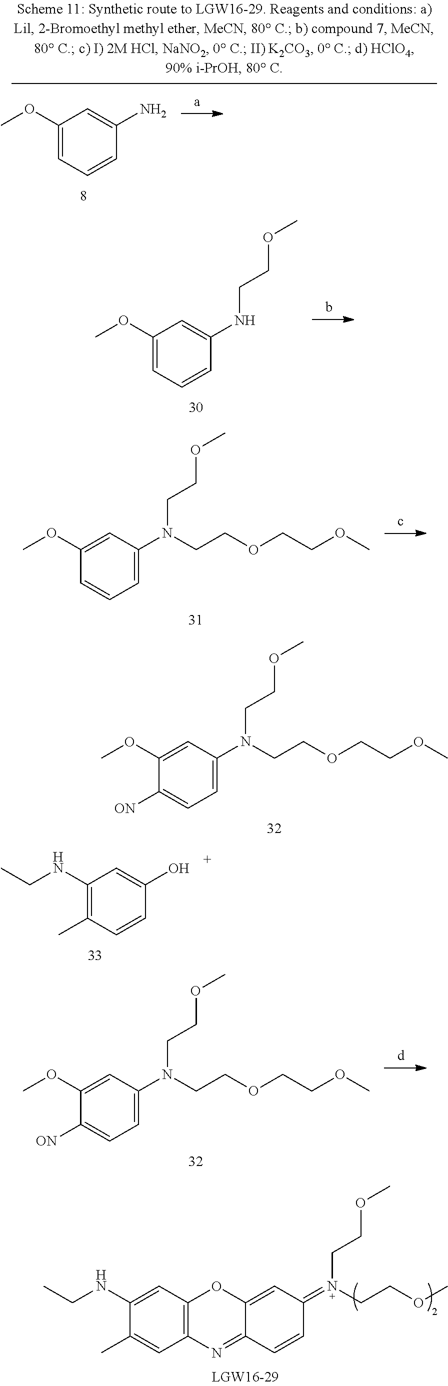

3-methoxy-N-(2-methoxyethyl) aniline (30): Compound 8 (1 g, 8.12 mmol), 2-bromoethyl methyl ether (0.915 mL, 9.74 mmol), Lil (543 mg, 4.06 mmol), and K₂CO₃ (1.69 g, 12.2 mmol) were suspended in anhydrous MeCN (20 mL) under N₂. The reaction mixture was then heated to 80° C. and stirred overnight before cooled to rt and diluted with DCM (50 mL). The solid was removed via vacuum filtration through Celite. The solvent was removed using a rotary evaporator and the residue was purified by flash column chromatography using a gradient of DCM (50-100%) in hexanes as eluent afforded compound 30 (1.13 g, 73%) as a clear oil.

3-methoxy-N-(2-(2-methoxyethoxy)ethyl)-N-(2-methoxyethyl) aniline (31): Compound 30 (0.68 g, 3.75 mmol), compound 7 (1.29 g, 5.63 mmol), and K₂CO₃ (1.04 g, 7.50 mmol) were suspended in anhydrous MeCN (15 mL) under N₂. The reaction mixture was then heated to 80° C. and stirred for 12 h prior to the second addition of compound 7 (0.43 g, 1.88 mmol). The reaction mixture was stirred for an additional 12 h before cooled to rt and diluted with DCM (30 mL). The solid was removed via vacuum filtration through Celite. The solvent was removed using a rotary evaporator and the residue was purified by flash column chromatography using a gradient of EtOAc (5-30%) in hexanes as eluent afforded compound 31 (0.82 g, 77%) as a light-yellow oil.

3-methoxy-N-(2-(2-methoxyethoxy)ethyl)-N-(2-methoxyethyl)-4-nitrosoaniline (32): Compound 31 (0.36 g, 1.27 mmol) was dissolved in an ice-cold 2 M HCl solution (5 mL). To this solution was added NaNO₂ (106 mg, 1.52 mmol) portion-wise over 0.5 h while the temperature of the solution was maintained below 5° C. The reaction mixture was stirred for an additional 2 h in the ice bath. The solution was carefully basified with solid K₂CO₃ until the pH value of the solution had risen above 8. The aqueous solution was then extracted with DCM (3×25 mL), and the combined organic layers were washed with brine (25 mL) before dried over anhydrous Na₂SO₄ and concentrated in vacuo. The title compound was obtained (0.34 g, 85%) as a dark green viscous oil, which was used for the next step without further purification.

(E)-N-(7-(ethylamino)-8-methyl-3H-phenoxazin-3-ylidene)-2-methoxy-N-(2-(2-methoxyethoxy)ethyl) ethan-1-aminium (LGW16-29): A 2-dram vial was charged with compound 33 (50 mg, 0.33 mmol), compound 32 (108 mg, 0.35 mmol), and a magnetic stir bar. To the vial was added a solution of i-PrOH/H₂O (9:1, 3 mL) and HClO₄ (70%, 30 μL). The resulting solution was stirred at 90° C. for 4 h to give a dark-blue solution that was concentrated under reduced pressure. The residue was purified by flash column chromatography using a gradient of CHCl₃ and MeOH containing 0.5% formic acid (gradient, 2-15% of MeOH in CHCl₃) to give compound LGW16-29 (80 mg, 58%).

Tables 1A and 1B: Current Small Molecule Organic Fluorophores with Nerve Specificity & Potential for a Near Infrared Fluorophore Upon Derivatization.

TABLE 1A

| | | | # |
|---|---|---|---|
| | Excita- | Emis- | Nerve- |
| | tion | sion | specific |
| 1. Nerve-specific fluorophore | (nm) | (nm) | probes |
| 2. Nerve-specific peptide | 492, 646 | 517, 662 | 2 |
| 3. Stilbene derivatives | 350, 363 | 415, 419 | 2 |
| 4. Coumarin fluorophore | 407 | 551 | 1 |
| 5. Distyryl benzene (DSB) derivatives | 247-396 | 431-656 | 242 |
| 6. Styryl pyridinium (FM) derivatives | 490-519 | 628-815 | 8 |
| 7. Oxazine fluorophore | 616, 625 | 635, 650 | 2 |
| 8. Tricarbocyanine (TCC) fluorophore | 760 | 800 | 1 |

TABLE 1B

| 1. Nerve-specific | | | Potential for NIR nerve- |
|---|---|---|---|
| fluorophore | Advantages | Disadvantages | specificity |
| 2. Nerve-specific | Peripheral | Too large for BNB | Low - conjugation to NIR |
| peptide | nerve | penetration, nonspecific | fluorophores possible, but |
| | specificity | skin & adipose signal | nerve SBR low due to |
| | | | nonspecific tissue |
| | | | accumulation |
| 3. Stilbene | Reported | UV excitation & | Low - Too few double bonds |
| derivatives | myelin | emission, nerve- | to reach NIR excitation or |
| | specificity in | specificity unknown | emission |
| | brain | | |
| 4. Coumarin | Reported | Fluorescence emission | Low - Too few double bonds |
| fluorophore | myelin | solvent dependent, | to reach NIR excitation or |
| | specificity in | nerve specificity | emission |
| | brain | unknown | |
| 5. Distyryl | Highlights all | Fluorescence emission | Medium - SAR for nerve- |
| benzene (DSB) | nerves in CNS | solvent dependent, | specificity known, sufficient |
| derivatives | & PNS | nerve specificity | double bonds to reach NIR |
| | | unknown | emission, NIR excitation |
| | | | challenging |
| 6. Styryl | NIR emission + | Visible excitation, | Medium - Direct |
| pyridinium (FM) | some nerve | limited nerve specificity | administration may provide |
| derivatives | specificity | following systemic | nerve-specificity, NIR |
| | | administration (DRG & | excitation may be synthetically |
| | | TG only) | available |
| 7. Oxazine | Highlights all | Excitation & emission | High - Current excitation & |
| fluorophore | nerves in CNS | not yet in the NIR region | emission close to NIR with |
| | & PNS when | (650-900 nm) | strong possibility for synthetic |
| | administered | | tuning, highly nerve-specific |
| | systemically | | |
| 8. | NIR excitation | No nerve partitioning | Medium - NIR excitation & |
| Tricarbocyanine | & emission, | following systemic | emission, may be synthetically |
| (TCC) | reported | administration, no nerve | tunable to create improved |
| fluorophore | myelin | accumulation after 4 | nerve-specificity |
| | specificity in | hrs + rapid clearance | |
| | the brain | | |

### REFERENCES

1. Chance, B. Near-infrared images using continuous, phase-modulated, and pulsed light with quantitation of blood and blood oxygenation. Annals of the New York Academy of Sciences 838, 29-45 (1998).
2. Vahrmeijer, A. L., Hutteman, M., van der Vorst, J. R., van de Velde, C. J. & Frangioni, J. V. Image-guided cancer surgery using near-infrared fluorescence. Nature reviews. Clinical oncology 10, 507-518 (2013).
3. Frangioni, J. V. In vivo near-infrared fluorescence imaging. Current opinion in chemical biology 7, 626-634 (2003).
4. Gibbs, S. L. Near infrared fluorescence for image-guided surgery. Quantitative imaging in medicine and surgery 2, 177-187 (2012).
5. Whitney, M. A. et al. Fluorescent peptides highlight peripheral nerves during surgery in mice. Nature biotechnology 29, 352-356 (2011).
6. Wu, C. et al. Molecular probes for imaging myelinated white matter in CNS. Journal of medicinal chemistry 51, 6682-6688 (2008).
7. Wang, C. et al. In situ fluorescence imaging of myelination. The journal of histochemistry and cytochemistry: official journal of the Histochemistry Society 58, 611-621 (2010).
8. Stankoff, B. et al. Imaging of CNS myelin by positron-emission tomography. Proceedings of the National Academy of Sciences of the United States of America 103, 9304-9309 (2006).
9. Cotero, V. E. et al. Intraoperative fluorescence imaging of peripheral and central nerves through a myelin-selective contrast agent. Molecular imaging and biology: MIB: the official publication of the Academy of Molecular Imaging 14, 708-717 (2012).
10 Gibbs, S. L. et al. Structure-activity relationship of nerve-highlighting fluorophores. PloS one 8, e73493 (2013).
11. Gibbs-Strauss, S. L. et al. Nerve-highlighting fluorescent contrast agents for image-guided surgery. Molecular imaging 10, 91-101 (2011).
12 Meyers, J. R. et al. Lighting up the senses: FM1-43 loading of sensory cells through nonselective ion channels. The Journal of neuroscience: the official journal of the Society for Neuroscience 23, 4054-4065 (2003).
13. Gibbs-Strauss, S. L. et al. Molecular imaging agents specific for the annulus fibrosus of the intervertebral disk. Molecular imaging 9, 128-140 (2010).
14. Park, M. H. et al. Prototype nerve-specific near-infrared fluorophores. Theranostics 4, 823-833 (2014).
15 Wang, C. et al. Longitudinal near-infrared imaging of myelination. The Journal of neuroscience: the official journal of the Society for Neuroscience 31, 2382-2390 (2011).

What is claimed:

1. A compound of Formula (I): 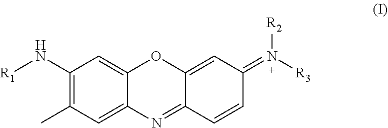
wherein:
R₁ is selected from the group of —X₁, —(CH₂)ₙ₁—SO₃; —(CH₂)ₙ₁—N⁺(CH₃)₃, —CH₂—CH₂—O—X₁, —CH₂—CH₂—O—[CH₂—CH₂—O]ₙ₄—X₁, —CH₂—CH₂—CH₂—O—X₁, and —CH₂—CH₂—CH₂—O—[CH₂—CH₂—CH₂—O]ₙ₅—X₁; 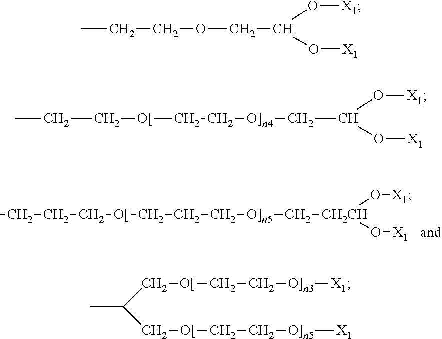
R₂ and R₃ are each independently selected from the group of methyl, ethyl, n-propyl, isopropyl, —(CH₂)ₙ₁—SO₃; —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; 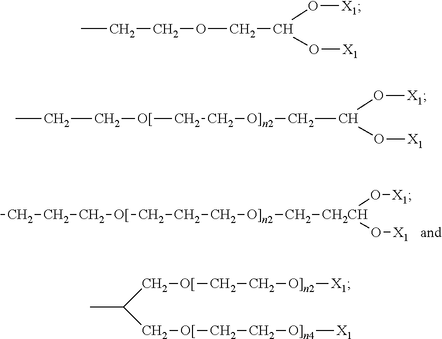
X₁ in each instance is independently selected from the group of C₁-C₆ straight or branched alkyl, C₂-C₆ straight or branched alkenyl, C₁-C₆ straight or branched alkynyl, and —Si(C₁-C₄ alkyl)₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
n5 is an integer independently selected in each instance from the group of 1, 2, 3, 4, 5, 6, 7, 8, 9, and 10;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

2. The compound of claim 1, having the Formula (II): 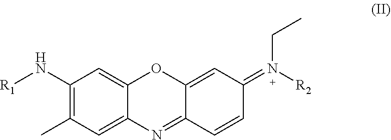
wherein:
R₁ and R₂ are each independently selected from the group of —(CH₂)ₙ₁—SO₃, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
n1 is an integer independently selected in each instance from the group of 1, 2, 3, and 4;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8.

3. The compound of claim 1, having the Formula (III): 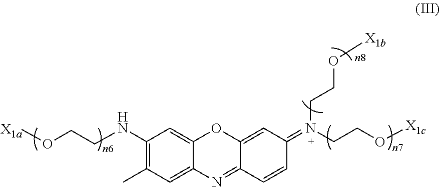
wherein:
X₁ₐ, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6, n7, and n8 are each an integer independently selected in each instance from the group of 1, 2, 3, 4, and 5.

4. The compound of claim 1, having Formula (IV): 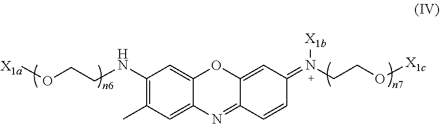
wherein:
X₁ₐ, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6 and n7 are each an integer independently selected in each instance from the group of 1, 2, 3, 4, and 5.

5. The compound of claim 1, having the Formula (V): 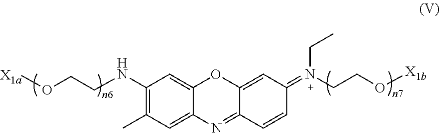
wherein:
X₁ₐ and X_{1b} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6 and n7 are each an integer independently selected in each instance from the group of 1, 2, 3, 4, and 5.

6. The compound of claim 1, having Formula (VI): 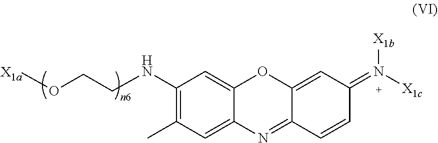
X₁ₐ, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6 is an integer selected from the group of 1, 2, 3, 4, and 5.

7. The compound of claim 1, having Formula (VII): 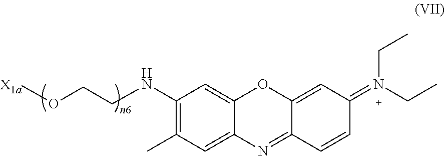
X₁ₐ is selected from the group of C₁-C₆ straight or branched alkyl, C₂-C₆ straight or branched alkenyl, C₁-C₆ straight or branched alkynyl, and —Si(C₁-C₄ alkyl)₃; and
n6 is an integer selected from the group of 1, 2, 3, 4, and 5.

8. The compound of claim 1, having Formula (VII): 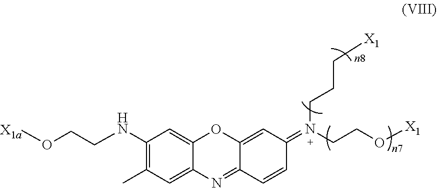
wherein:
X₁ₐ, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n7 and n8 are each an integer independently selected in each instance from the group of 1, 2, 3, 4, and 5.

9. The compound of claim 1, having Formula (IX): 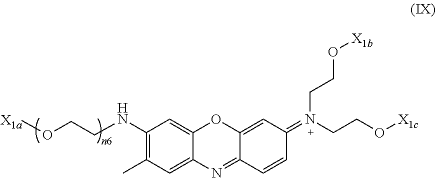
wherein:
X₁ₐ, X_{1b}, and X_{1c} are each independently selected from the group of C₁-C₆ straight or branched alkyl, C₂-C₆ straight or branched alkenyl, C₁-C₆ straight or branched alkynyl, and —Si(C₁-C₄ alkyl)₃; and
n6 is an integer independently selected from the group of 1, 2, 3, 4, and 5.

10. The compound of claim 1, having Formula (X): 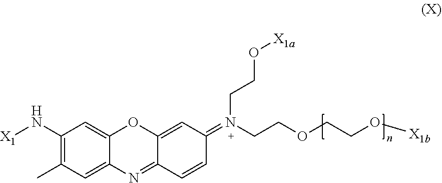
wherein:
n is an integer selected from 2, 3, 4, and 5;
X₁ is C₁-C₆ alkyl; and
X₁ₐ and X_{1b} are each selected independently from the group of C₁-C₆ straight or branched alkyl, C₂-C₆ straight or branched alkenyl, C₁-C₆ straight or branched alkynyl, and —Si(C₁-C₄ alkyl)₃.

11. The compound of claim 1 selected from the group of:

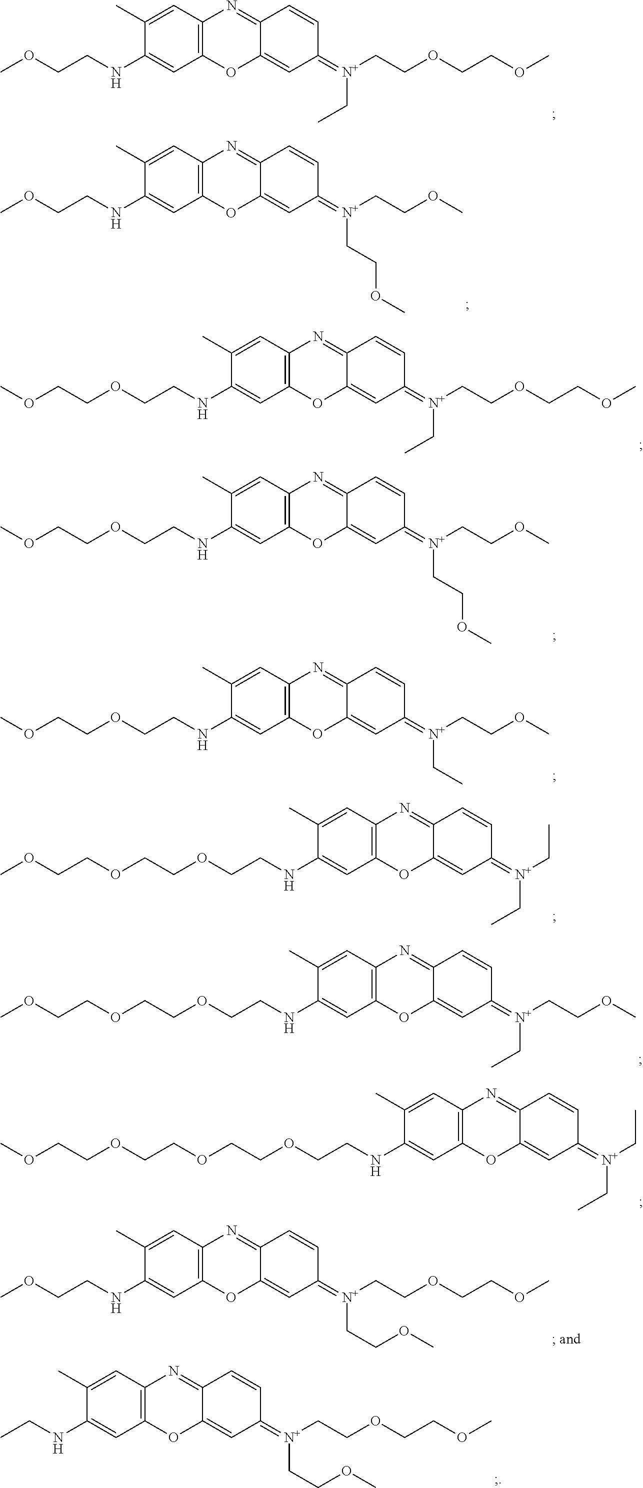

12. A composition comprising a compound of claim 1 and a pharmaceutically acceptable carrier or excipient.

13. A composition comprising a compound of claim 1 and buffered saline.

14. A method of detecting nerves intraoperatively in a subject undergoing surgery, the method comprising the steps of:
a) systemically administering an effective amount of a composition comprising a compound of claim 1 to the subject before or during surgery to form a stained tissue; and
b) imaging the stained tissue in the subject, thereby detecting nerves intraoperatively in the subject.
